(19) United States
(12) Patent Application Publication
(10) Pub. No.: US 2007/0219459 A1
(43) Pub. Date: Sep. 20, 2007

(54) Biopsy Devices, Methods for Using, and Methods for Making

(75) Inventor: Adam L. Cohen, Los Angeles, CA (US)

Correspondence Address: MICROFABRICA INC.;ATT: DENNIS R. SMALLEY, 7911 HASKELL AVENUE, VAN NUYS, CA, 91406, US

(73) Assignee: Microfabrica Inc.

(21) Appl. No.: 11/696,722

(22) Filed: Apr. 4, 2007

Related U.S. Application Data
(63) Continuation-in-part of application No. 11/582,049, filed on Oct. 16, 2006.
(60) Provisional application No. 60/789,378, filed on Apr. 4, 2006.
(60) Provisional application No. 60/726,794, filed on Oct. 14, 2005.

(51) Int. Cl. A61B 10/00

(52) U.S. Cl. 600/564; 600/567

(57) ABSTRACT

Embodiments of invention are directed to devices, and methods of forming them, that can be used for removal of small volumes of tissue from the body, as is required for biopsy procedures. The procedures may be of the image-guided or non image-guided type. The devices described herein are applicable to biopsy of many types of tissue, particularly soft tissue. Applications include biopsy of the intestines, stomach, breast, pancreas, lung, liver, brain, skin, prostate, thyroid, heart, and other organs and tissues. Embodiments are directed to devices formed from a plurality of adhered layers of deposited materials.

### RELATED APPLICATIONS

This application claims benefit of U.S. Provisional Patent Application No. 60/789,378, filed Apr. 4, 2006, and is a continuation-in-part of U.S. patent application Ser. No. 11/582,049, filed Oct. 16, 2006 which in turn claims benefit of U.S. Provisional Patent Application No. 60/726,794, filed Oct. 14, 2005. Each of these applications is incorporated herein by reference as if set forth in full herein.

### FIELD OF THE INVENTION

The present invention relates generally to micro-scale and meso-scale medical devices or instruments and particularly to micro-scale or meso-scale surgical tools, methods for using such devices, and methods for making such devices and even more particularly to medical devices that can be used for removal of small volumes of tissue from the body, as is required for biopsy procedures. In some embodiments the devices are fabricated from a plurality of substantially planar layers which are adhered to previously formed layers as material forming each layer is deposited and wherein each layer is formed from at least one structural material and at least one sacrificial material.

### BACKGROUND OF THE INVENTION

An electrochemical fabrication technique for forming three-dimensional structures from a plurality of adhered layers is being commercially pursued by Microfabrica Inc. (formerly MEMGen® Corporation) of Van Nuys, Calif. under the name EFAB™.

Various electrochemical fabrication techniques were described in U.S. Patent Ser. No. 6,027,630, issued on Feb. 22, 2000 to Adam Cohen. Some embodiments of this electrochemical fabrication technique allows the selective deposition of a material using a mask that includes a patterned conformable material on a support structure that is independent of the substrate onto which plating will occur. When desiring to perform an electrodeposition using the mask, the conformable portion of the mask is brought into contact with a substrate, but not adhered or bonded to the substrate, while in the presence of a plating solution such that the contact of the conformable portion of the mask to the substrate inhibits deposition at selected locations. For convenience, these masks might be generically called conformable contact masks; the masking technique may be generically called a conformable contact mask plating process. More specifically, in the terminology of Microfabrica Inc. such masks have come to be known as INSTANT MASKS™ and the process known as INSTANT MASKING™ or INSTANT MASK™ plating. Selective depositions using conformable contact mask plating may be used to form single selective deposits of material or may be used in a process to form multi-layer structures. The teachings of the '630 patent are hereby incorporated herein by reference as if set forth in full herein. Since the filing of the patent application that led to the above noted patent, various papers about conformable contact mask plating (i.e. INSTANT MASKING) and electrochemical fabrication have been published:
(1) A. Cohen, G. Zhang, F. Tseng, F. Mansfeld, U. Frodis and P. Will, “EFAB: Batch production of functional, fully-dense metal parts with micro-scale features”, Proc. 9th Solid Freeform Fabrication, The University of Texas at Austin, p161, Aug. 1998.
(2) A. Cohen, G. Zhang, F. Tseng, F. Mansfeld, U. Frodis and P. Will, “EFAB: Rapid, Low-Cost Desktop Micromachining of High Aspect Ratio True 3-D MEMS”, Proc. 12th IEEE Micro Electro Mechanical Systems Workshop, IEEE, p244, Jan. 1999.
(3) A. Cohen, “3-D Micromachining by Electrochemical Fabrication”, Micromachine Devices, Mar. 1999.
(4) G. Zhang, A. Cohen, U. Frodis, F. Tseng, F. Mansfeld, and P. Will, “EFAB: Rapid Desktop Manufacturing of True 3-D Microstructures”, Proc. 2nd International Conference on Integrated MicroNanotechnology for Space Applications, The Aerospace Co., Apr. 1999.
(5) F. Tseng, U. Frodis, G. Zhang, A. Cohen, F. Mansfeld, and P. Will, “EFAB: High Aspect Ratio, Arbitrary 3-D Metal Microstructures using a Low-Cost Automated Batch Process”, 3rd International Workshop on High Aspect Ratio MicroStructure Technology (HARMST'99), Jun. 1999.
(6) A. Cohen, U. Frodis, F. Tseng, G. Zhang, F. Mansfeld, and P. Will, “EFAB: Low-Cost, Automated Electrochemical Batch Fabrication of Arbitrary 3-D Microstructures”, Micromachining and Microfabrication Process Technology, SPIE 1999 Symposium on Micromachining and Microfabrication, Sep. 1999.
(7) F. Tseng, G. Zhang, U. Frodis, A. Cohen, F. Mansfeld, and P. Will, “EFAB: High Aspect Ratio, Arbitrary 3-D Metal Microstructures using a Low-Cost Automated Batch Process”, MEMS Symposium, ASME 1999 International Mechanical Engineering Congress and Exposition, Nov., 1999.
(8) A. Cohen, “Electrochemical Fabrication (EFABTM)”, Chapter 19 of The MEMS Handbook, edited by Mohamed Gad-EI-Hak, CRC Press, 2002.
(9) Microfabrication—Rapid Prototyping's Killer Application”, pages 1-5 of the Rapid Prototyping Report, CAD/CAM Publishing, Inc., Jun. 1999.

The disclosures of these nine publications are hereby incorporated herein by reference as if set forth in full herein.

An electrochemical deposition for forming multilayer structures may be carried out in a number of different ways as set forth in the above patent and publications. In one form, this process involves the execution of three separate operations during the formation of each layer of the structure that is to be formed:
1. Selectively depositing at least one material by electrodeposition upon one or more desired regions of a substrate. Typically this material is either a structural material or a sacrificial material.
2. Then, blanket depositing at least one additional material by electrodeposition so that the additional deposit covers both the regions that were previously selectively deposited onto, and the regions of the substrate that did not receive any previously applied selective depositions. Typically this material is the other of a structural material or a sacrificial material.
3. Finally, planarizing the materials deposited during the first and second operations to produce a smoothed surface of a first layer of desired thickness having at least one region containing the at least one material and at least one region containing at least the one additional material.

After formation of the first layer, one or more additional layers may be formed adjacent to an immediately preceding layer and adhered to the smoothed surface of that preceding layer. These additional layers are formed by repeating the first through third operations one or more times wherein the formation of each subsequent layer treats the previously formed layers and the initial substrate as a new and thickening substrate.

Once the formation of all layers has been completed, at least a portion of at least one of the materials deposited is generally removed by an etching process to expose or release the three-dimensional structure that was intended to be formed. The removed material is a sacrificial material while the material that forms part of the desired structure is a structural material.

The preferred method of performing the selective electrodeposition involved in the first operation is by conformable contact mask plating. In this type of plating, one or more conformable contact (CC) masks are first formed. The CC masks include a support structure onto which a patterned conformable dielectric material is adhered or formed. The conformable material for each mask is shaped in accordance with a particular cross-section of material to be plated (the pattern of conformable material is complementary to the pattern of material to be deposited). At least one CC mask is used for each unique cross-sectional pattern that is to be plated.

The support for a CC mask is typically a plate-like structure formed of a metal that is to be selectively electroplated and from which material to be plated will be dissolved. In this typical approach, the support will act as an anode in an electroplating process. In an alternative approach, the support may instead be a porous or otherwise perforated material through which deposition material will pass during an electroplating operation on its way from a distal anode to a deposition surface. In either approach, it is possible for multiple CC masks to share a common support, i.e. the patterns of conformable dielectric material for plating multiple layers of material may be located in different areas of a single support structure. When a single support structure contains multiple plating patterns, the entire structure is referred to as the CC mask while the individual plating masks may be referred to as “submasks”. In the present application such a distinction will be made only when relevant to a specific point being made.

In preparation for performing the selective deposition of the first operation, the conformable portion of the CC mask is placed in registration with and pressed against a selected portion of (1) the substrate, (2) a previously formed layer, or (3) a previously deposited portion of a layer on which deposition is to occur. The pressing together of the CC mask and relevant substrate occur in such a way that all openings, in the conformable portions of the CC mask contain plating solution. The conformable material of the CC mask that contacts the substrate acts as a barrier to electrodeposition while the openings in the CC mask that are filled with electroplating solution act as pathways for transferring material from an anode (e.g. the CC mask support) to the non-contacted portions of the substrate (which act as a cathode during the plating operation) when an appropriate potential and/or current are supplied.

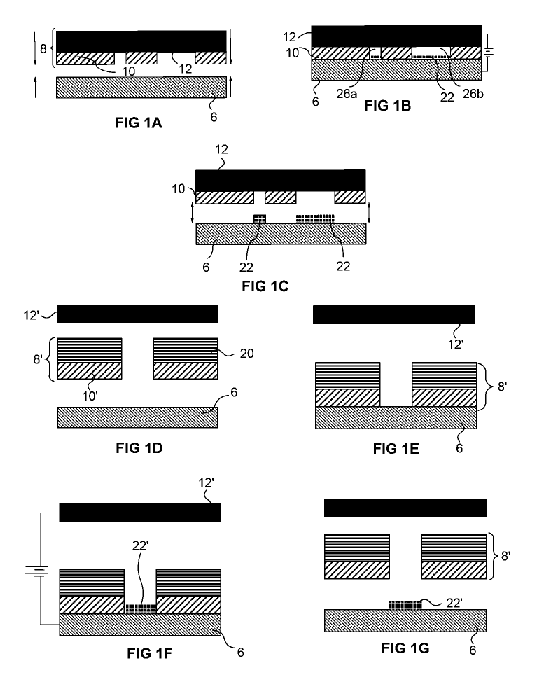

An example of a CC mask and CC mask plating are shown in FIGS. 1A-1C. FIG. 1A shows a side view of a CC mask 8 consisting of a conformable or deformable (e.g. elastomeric) insulator 10 patterned on an anode 12. The anode has two functions. One is as a supporting material for the patterned insulator 10 to maintain its integrity and alignment since the pattern may be topologically complex (e.g., involving isolated “islands” of insulator material). The other function is as an anode for the electroplating operation. FIG. 1A also depicts a substrate 6, separated from mask 8, onto which material will be deposited during the process of forming a layer. CC mask plating selectively deposits material 22 onto substrate 6 by simply pressing the insulator against the substrate then electrodepositing material through apertures 26a and 26b in the insulator as shown in FIG. 1B. After deposition, the CC mask is separated, preferably non-destructively, from the substrate 6 as shown in FIG. 1C.

The CC mask plating process is distinct from a “through-mask” plating process in that in a through-mask plating process the separation of the masking material from the substrate would occur destructively. Furthermore in a through mask plating process, opening in the masking material are typically formed while the masking material is in contact with and adhered to the substrate. As with through-mask plating, CC mask plating deposits material selectively and simultaneously over the entire layer. The plated region may consist of one or more isolated plating regions where these isolated plating regions may belong to a single structure that is being formed or may belong to multiple structures that are being formed simultaneously. In CC mask plating as individual masks are not intentionally destroyed in the removal process, they may be usable in multiple plating operations.

Another example of a CC mask and CC mask plating is shown in FIGS. 1D-1G. FIG. 1D shows an anode 12′ separated from a mask 8′ that includes a patterned conformable material 10′ and a support structure 20. FIG. 1D also depicts substrate 6 separated from the mask 8′. FIG. 1E illustrates the mask 8′ being brought into contact with the substrate 6. FIG. 1F illustrates the deposit 22′ that results from conducting a current from the anode 12′ to the substrate 6. FIG. 1G illustrates the deposit 22′ on substrate 6 after separation from mask 8′. In this example, an appropriate electrolyte is located between the substrate 6 and the anode 12′ and a current of ions coming from one or both of the solution and the anode are conducted through the opening in the mask to the substrate where material is deposited. This type of mask may be referred to as an anodeless INSTANT MASK™ (AIM) or as an anodeless conformable contact (ACC) mask.

Unlike through-mask plating, CC mask plating allows CC masks to be formed completely separate from the substrate on which plating is to occur (e.g. separate from a three-dimensional (3D) structure that is being formed). CC masks may be formed in a variety of ways, for example, using a photolithographic process. All masks can be generated simultaneously, e.g. prior to structure fabrication rather than during it. This separation makes possible a simple, low-cost, automated, self-contained, and internally-clean “desktop factory” that can be installed almost anywhere to fabricate 3D structures, leaving any required clean room processes, such as photolithography to be performed by service bureaus or the like.

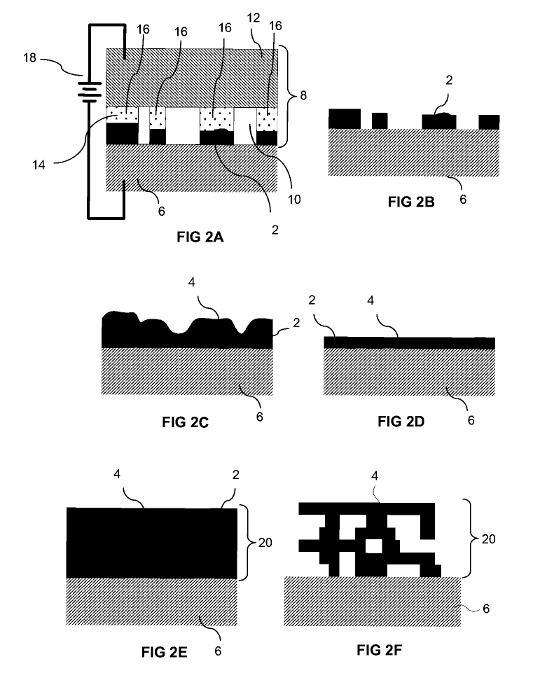

An example of the electrochemical fabrication process discussed above is illustrated in FIGS. 2A-2F. These figures show that the process involves deposition of a first material 2 which is a sacrificial material and a second material 4 which is a structural material. The CC mask 8, in this example, includes a patterned conformable material (e.g. an elastomeric dielectric material) 10 and a support 12 which is made from deposition material 2. The conformal portion of the CC mask is pressed against substrate 6 with a plating solution 14 located within the openings 16 in the conformable material 10. An electric current, from power supply 18, is then passed through the plating solution 14 via (a) support 12 which doubles as an anode and (b) substrate 6 which doubles as a cathode. FIG. 2A illustrates that the passing of current causes material 2 within the plating solution and material 2 from the anode 12 to be selectively transferred to and plated on the substrate 6. After electroplating the first deposition material 2 onto the substrate 6 using CC mask 8, the CC mask 8 is removed as shown in FIG. 2B. FIG. 2C depicts the second deposition material 4 as having been blanket-deposited (i.e. non-selectively deposited) over the previously deposited first deposition material 2 as well as over the other portions of the substrate 6. The blanket deposition occurs by electroplating from an anode (not shown), composed of the second material, through an appropriate plating solution (not shown), and to the cathode/substrate 6. The entire two-material layer is then planarized to achieve precise thickness and flatness as shown in FIG. 2D. After repetition of this process for all layers, the multi-layer structure 20 formed of the second material 4 (i.e. structural material) is embedded in first material 2 (i.e. sacrificial material) as shown in FIG. 2E. The embedded structure is etched to yield the desired device, i.e. structure 20, as shown in FIG. 2F.

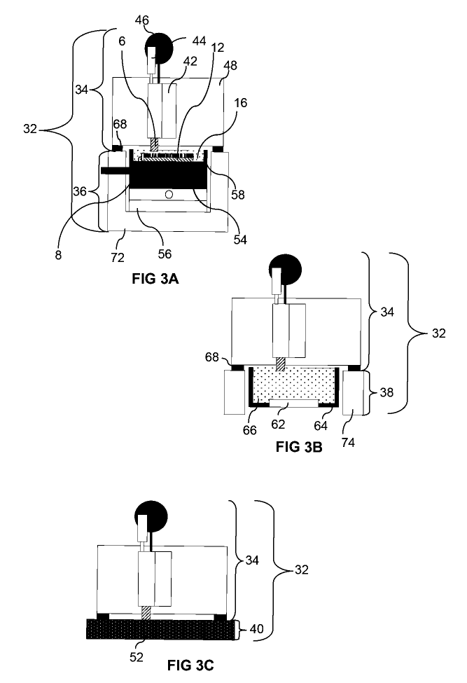

Various components of an exemplary manual electrochemical fabrication system 32 are shown in FIGS. 3A-3C. The system 32 consists of several subsystems 34, 36, 38, and 40. The substrate holding subsystem 34 is depicted in the upper portions of each of FIGS. 3A-3C and includes several components: (1) a carrier 48, (2) a metal substrate 6 onto which the layers are deposited, and (3) a linear slide 42 capable of moving the substrate 6 up and down relative to the carrier 48 in response to drive force from actuator 44. Subsystem 34 also includes an indicator 46 for measuring differences in vertical position of the substrate which may be used in setting or determining layer thicknesses and/or deposition thicknesses. The subsystem 34 further includes feet 68 for carrier 48 which can be precisely mounted on subsystem 36.

The CC mask subsystem 36 shown in the lower portion of FIG. 3A includes several components: (1) a CC mask 8 that is actually made up of a number of CC masks (i.e. submasks) that share a common support/anode 12, (2) precision X-stage 54, (3) precision Y-stage 56, (4) frame 72 on which the feet 68 of subsystem 34 can mount, and (5) a tank 58 for containing the electrolyte 16. Subsystems 34 and 36 also include appropriate electrical connections (not shown) for connecting to an appropriate power source (not shown) for driving the CC masking process.

The blanket deposition subsystem 38 is shown in the lower portion of FIG. 3B and includes several components: (1) an anode 62, (2) an electrolyte tank 64 for holding plating solution 66, and (3) frame 74 on which feet 68 of subsystem 34 may sit. Subsystem 38 also includes appropriate electrical connections (not shown) for connecting the anode to an appropriate power supply (not shown) for driving the blanket deposition process.

The planarization subsystem 40 is shown in the lower portion of FIG. 3C and includes a lapping plate 52 and associated motion and control systems (not shown) for planarizing the depositions.

In addition to teaching the use of CC masks for electrodeposition purposes, the '630 patent also teaches that the CC masks may be placed against a substrate with the polarity of the voltage reversed and material may thereby be selectively removed from the substrate. It indicates that such removal processes can be used to selectively etch, engrave, and polish a substrate, e.g., a plaque.

The '630 patent further indicates that the electroplating methods and articles disclosed therein allow fabrication of devices from thin layers of materials such as, e.g., metals, polymers, ceramics, and semiconductor materials. It further indicates that although the electroplating embodiments described therein have been described with respect to the use of two metals, a variety of materials, e.g., polymers, ceramics and semiconductor materials, and any number of metals can be deposited either by the electroplating methods therein, or in separate processes that occur throughout the electroplating method. It indicates that a thin plating base can be deposited, e.g., by sputtering, over a deposit that is insufficiently conductive (e.g., an insulating layer) so as to enable subsequent electroplating. It also indicates that multiple support materials (i.e. sacrificial materials) can be included in the electroplated element allowing selective removal of the support materials.

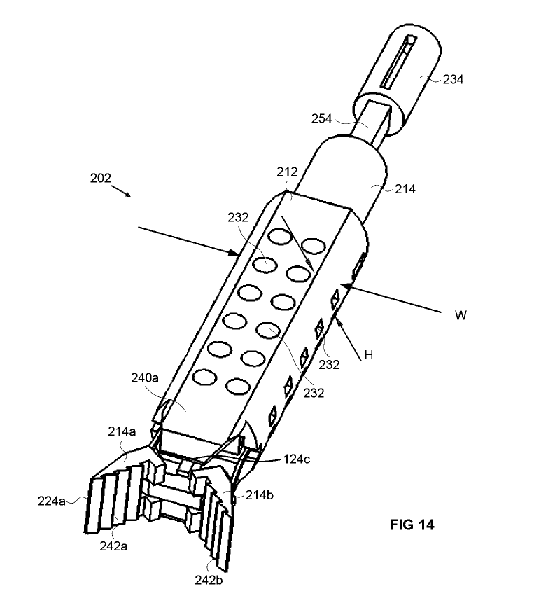

The '630 patent additionally teaches that the electroplating methods disclosed therein can be used to manufacture elements having complex microstructure and close tolerances between parts. An example is given with the aid of FIGS. 14A-14E of that patent. In the example, elements having parts that fit with close tolerances, e.g., having gaps between about 1-5 um, including electroplating the parts of the device in an unassembled, preferably pre-aligned, state and once fabricated. In such embodiments, the individual parts can be moved into operational relation with each other or they can simply fall together. Once together the separate parts may be retained by clips or the like.

Another method for forming microstructures from electroplated metals (i.e. using electrochemical fabrication techniques) is taught in U.S. Pat. No. 5,190,637 to Henry Guckel, entitled “Formation of Microstructures by Multiple Level Deep X-ray Lithography with Sacrificial Metal layers”. This patent teaches the formation of metal structure utilizing through mask exposures. A first layer of a primary metal is electroplated onto an exposed plating base to fill a void in a photoresist (the photoresist forming a through mask having a desired pattern of openings), the photoresist is then removed and a secondary metal is electroplated over the first layer and over the plating base. The exposed surface of the secondary metal is then machined down to a height which exposes the first metal to produce a flat uniform surface extending across both the primary and secondary metals. Formation of a second layer may then begin by applying a photoresist over the first layer and patterning it (i.e. to form a second through mask) and then repeating the process that was used to produce the first layer to produce a second layer of desired configuration. The process is repeated until the entire structure is formed and the secondary metal is removed by etching. The photoresist is formed over the plating base or previous layer by casting and patterning of the photoresist (i.e. voids formed in the photoresist) are formed by exposure of the photoresist through a patterned mask via X-rays or UV radiation and development of the exposed or unexposed areas.

The '637 patent teaches the locating of a plating base onto a substrate in preparation for electroplating materials onto the substrate. The plating base is indicated as typically involving the use of a sputtered film of an adhesive metal, such as chromium or titanium, and then a sputtered film of the metal that is to be plated. It is also taught that the plating base may be applied over an initial layer of sacrificial material (i.e. a layer or coating of a single material) on the substrate so that the structure and substrate may be detached if desired. In such cases after formation of the structure the sacrificial material forming part of each layer of the structure may be removed along the initial sacrificial layer to free the structure. Substrate materials mentioned in the '637 patent include silicon, glass, metals, and silicon with protected semiconductor devices. A specific example of a plating base includes about 150 angstroms of titanium and about 300 angstroms of nickel, both of which are sputtered at a temperature of 160° C. In another example it is indicated that the plating base may consist of 150 angstroms of titanium and 150 angstroms of nickel where both are applied by sputtering.

Electrochemical Fabrication provides the ability to form prototypes and commercial quantities of miniature objects, parts, structures, devices, and the like at reasonable costs and in reasonable times. In fact, Electrochemical Fabrication is an enabler for the formation of many structures that were hitherto impossible to produce. Electrochemical Fabrication opens the spectrum for new designs and products in many industrial fields. Even though Electrochemical Fabrication offers this new capability and it is understood that Electrochemical Fabrication techniques can be combined with designs and structures known within various fields to produce new structures, certain uses for Electrochemical Fabrication provide designs, structures, capabilities and/or features not known or obvious in view of the state of the art.

A need exists in various fields for miniature devices having improved characteristics, reduced fabrication times, reduced fabrication costs, simplified fabrication processes, greater versatility in device design, improved selection of materials, improved material properties, more cost effective and less risky production of such devices, and/or more independence between geometric configuration and the selected fabrication process.

### SUMMARY OF THE INVENTION

It is an object of some aspects of the invention to provide an improved method for forming micro-scale or meso-scale medical devices.

It is an object of some aspects of the invention to provide an improved meso-scale or microscale device for extracting a small specimen of tissue from the body of a patient.

It is an object of some aspects of the invention to provide improved methods of removing small amounts of tissue from the bodies of patients.

Other objects and advantages of various embodiments of the invention will be apparent to those of skill in the art upon review of the teachings herein. The various embodiments of the invention, set forth explicitly herein or otherwise ascertained from the teachings herein, may address one or more of the above objects alone or in combination, or alternatively may address some other object of the invention ascertained from the teachings herein. It is not necessarily intended that all objects be addressed by any single aspect of the invention even though that may be the case with regard to some aspects.

A first aspect of the invention provides a medical device for extracting a specimen of tissue from the body of a patient, including: (a) means for allowing a specimen to access an opening within the device; (b) means for holding the specimen within the opening; (c) means for separating the specimen from other tissue; and (d) means for releasing the specimen from the opening.

A second aspect of the invention provides a method for extracting a tissue specimen from a body of a patient, comprising: (a) locating an extraction device in proximity to the tissue to be sampled; (b) causing a specimen of tissue to enter an opening within the extraction device; (c) causing the device to hold the specimen within the opening; (d) causing the specimen to separate from other tissue; and (e) releasing the specimen from the opening.

Other aspects of the invention will be understood by those of skill in the art upon review of the teachings herein. Other aspects of the invention may involve combinations of the above noted aspects of the invention. These other aspects of the invention may provide various combinations of the aspects presented above as well as provide other configurations, structures, functional relationships, and processes that have not been specifically set forth above.

### BRIEF DESCRIPTION OF THE DRAEINGS:

FIGS. 1A-1C schematically depict side views of various stages of a CC mask plating process, while FIGS. 1D-G schematically depict a side views of various stages of a CC mask plating process using a different type of CC mask.

FIGS. 2A-2F schematically depict side views of various stages of an electrochemical fabrication process as applied to the formation of a particular structure where a sacrificial material is selectively deposited while a structural material is blanket deposited.

FIGS. 3A-3C schematically depict side views of various example subassemblies that may be used in manually implementing the electrochemical fabrication method depicted in FIGS. 2A-2F.

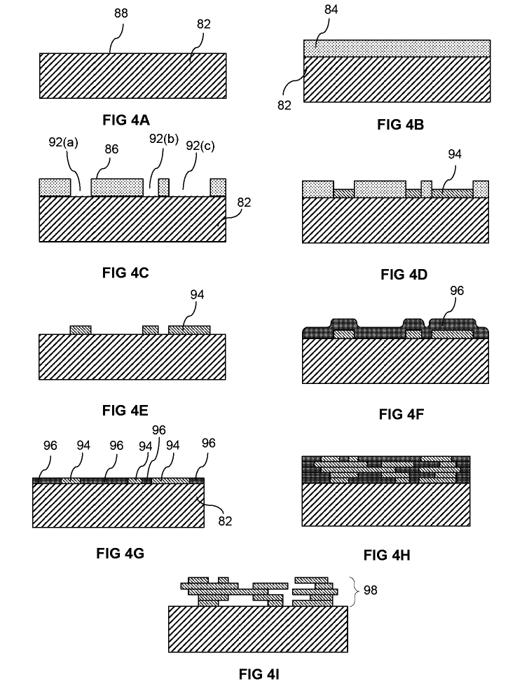

FIGS. 4A-4F schematically depict the formation of a first layer of a structure using adhered mask plating where the blanket deposition of a second material overlays both the openings between deposition locations of a first material and the first material itself

FIG. 4G depicts the completion of formation of the first layer resulting from planarizing the deposited materials to a desired level.

FIGS. 4H and 4I respectively depict the state of the process after formation of the multiple layers of the structure and after release of the structure from the sacrificial material.

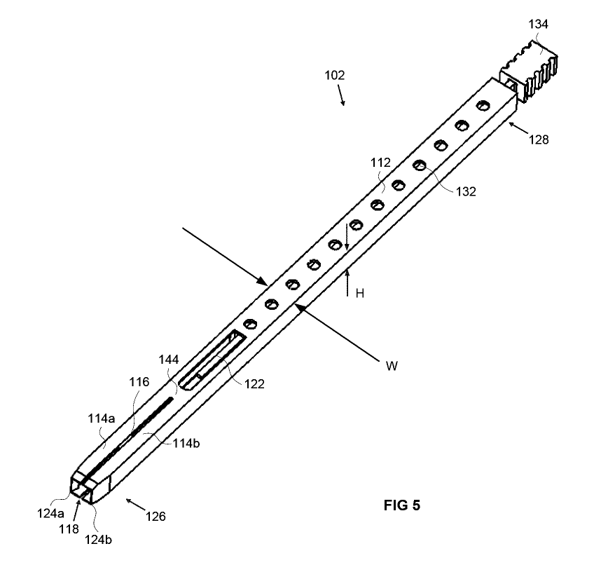

FIG. 5 provides a perspective overview of the device of the first embodiment.

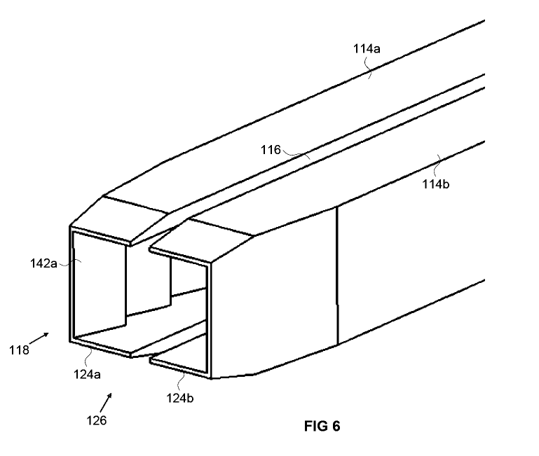

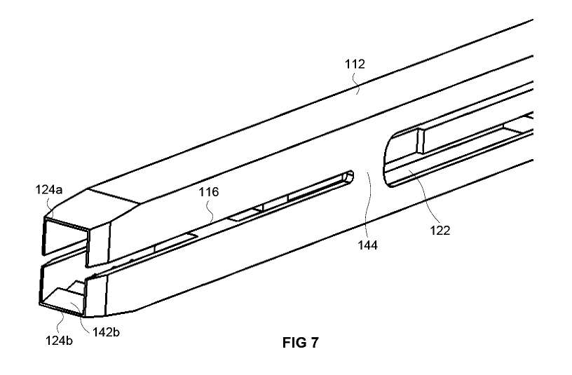

FIGS. 6-7 provide alternative, perspective views of the distal end of the device of FIG. 5

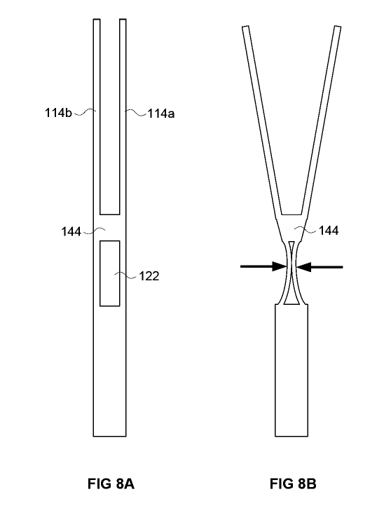

FIG. 8A and 8B illustrate schematically a closed and open state of the jaws of the device of FIG. 5.

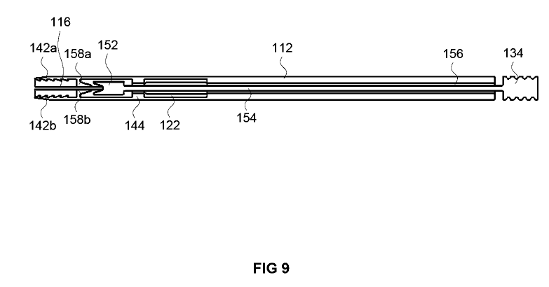

FIG. 9 provides a cross-sectional view through the device of FIG. 5 along the plane including the height and length of the device.

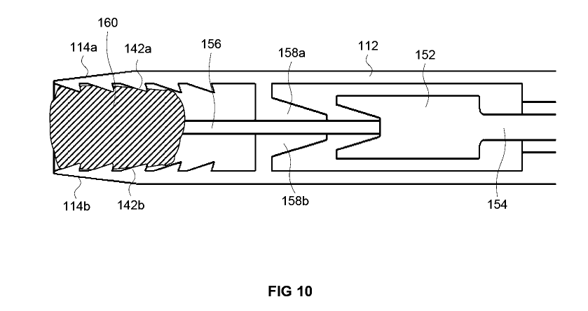

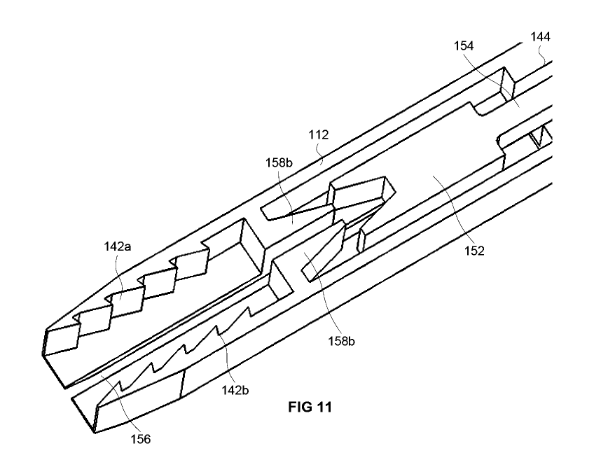

FIGS. 10-11 provide respectively a plan view of the distal end of the device of FIG. 5 holding a specimen and a perspective view of the distal end of the device.

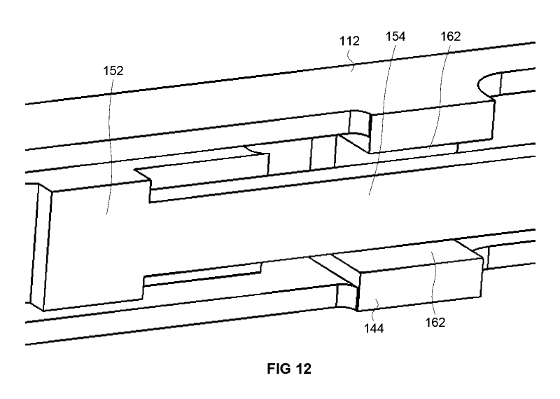

FIG. 12 provides a cross-sectional view of the device of FIG. 5 in the region of the fulcrum.

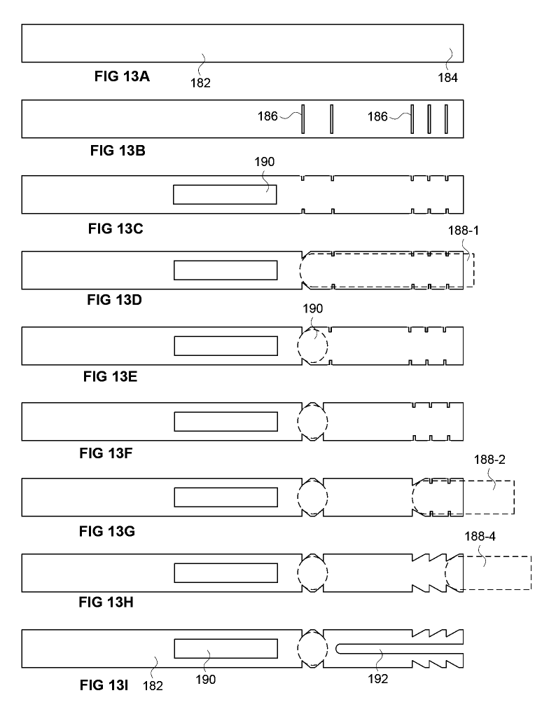

FIGS. 13A-13I illustrate an alternative to an EFAB™ process for producing a device like that of FIGS. 5-FIG. 12.

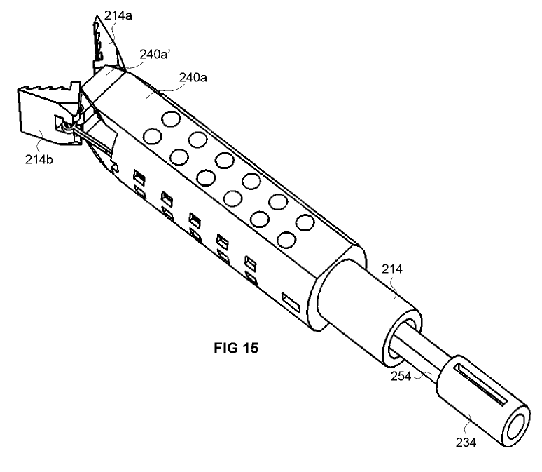

FIGS. 14 and 15 provide perspective view from distal and proximal ends, respectively, of a device according a second embodiment of the invention.

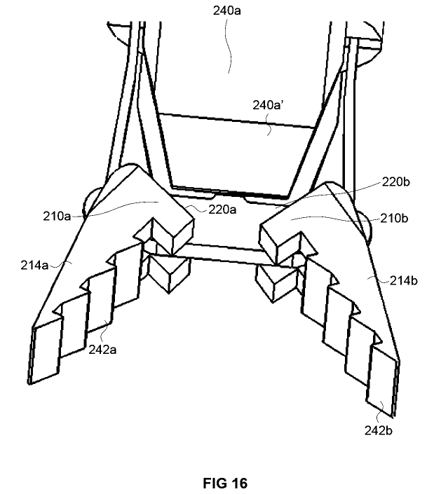

FIG. 16 provides a close up perspective view of the jaws of the device of FIGS. 14 and 15.

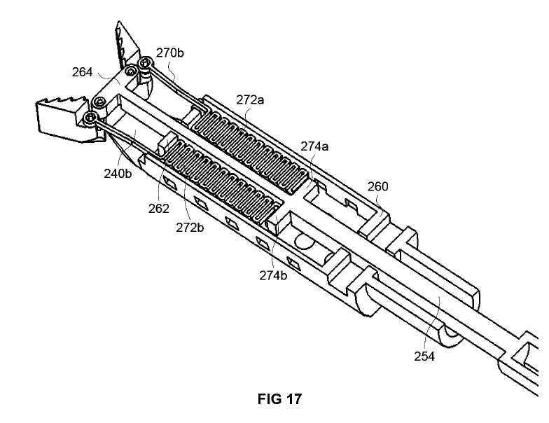

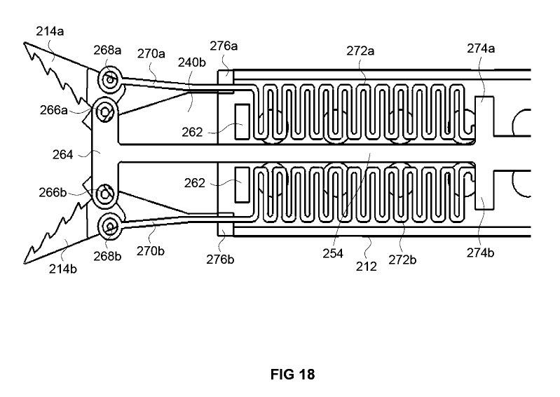

FIGS. 17 and 18 provide, respectively, perspective and plan cross-sectional views of the device of FIGS. 14-16 showing the internal springs and other details.

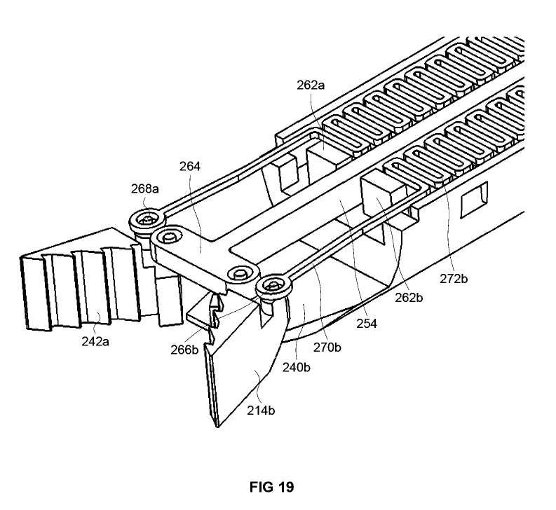

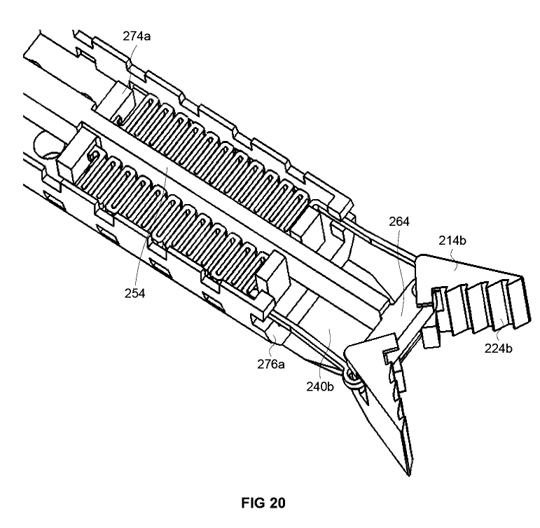

FIGS. 19 and 20 provide cross-sectional perspective views of the distal end of the device of FIGS. 14-18 taken at different cross-sectional levels with different perspectives.

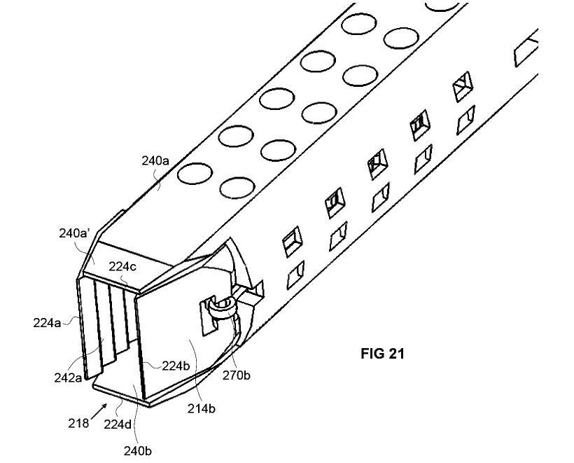

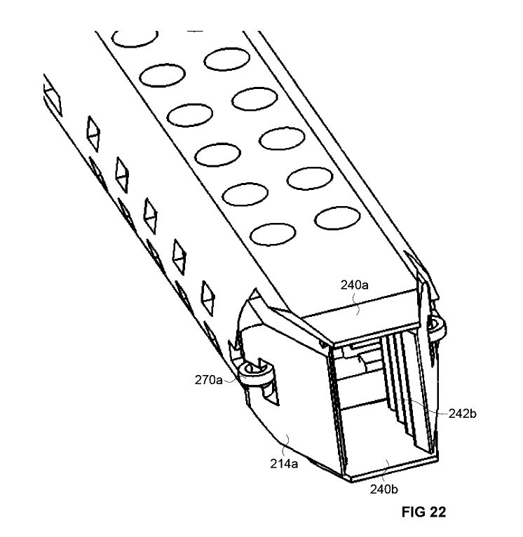

FIGS. 21 and 22 provide perspective views of the distal end of the device of FIGS. 14-20 taken from different perspectives.

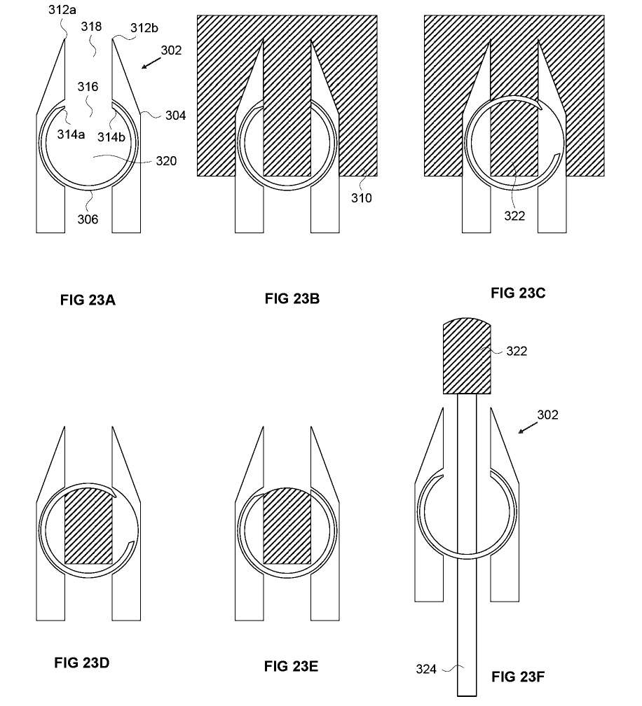

FIGS. 23A-23F provide a schematic representation of various stages of operation of a third embodiment of the invention.

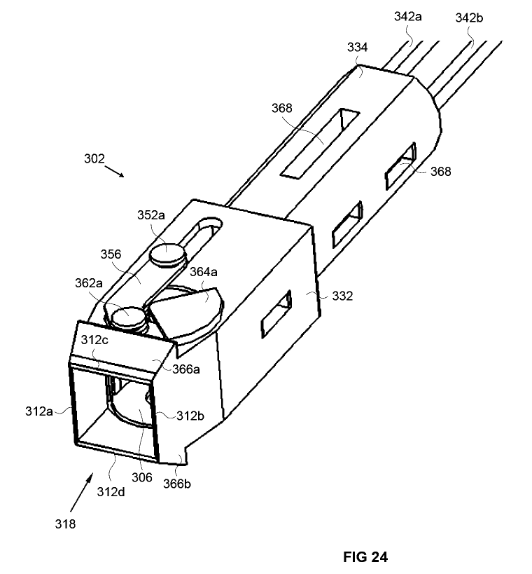

In FIG. 24 provides a perspective view of an example device of that is capable of being operated according to the method of use set forth in FIGS. 23A-23F.

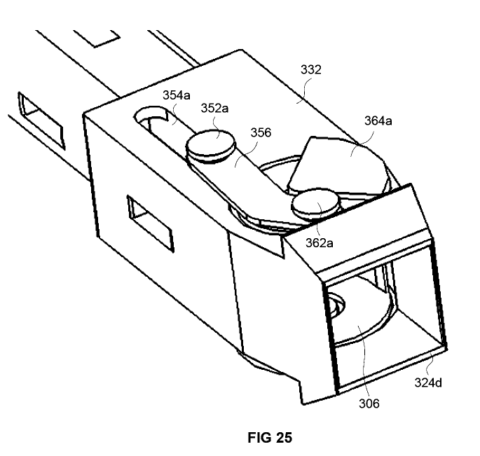

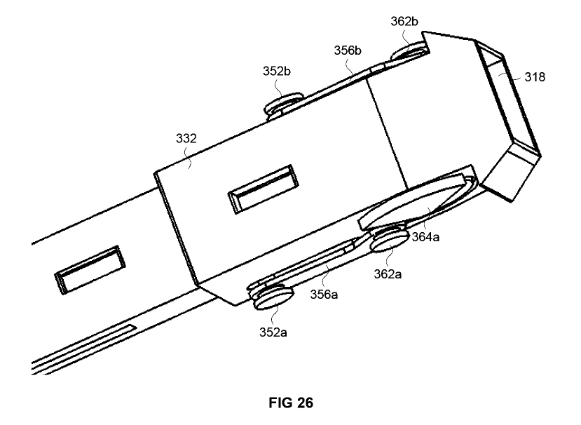

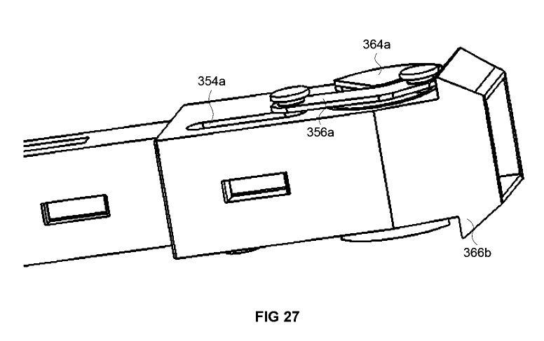

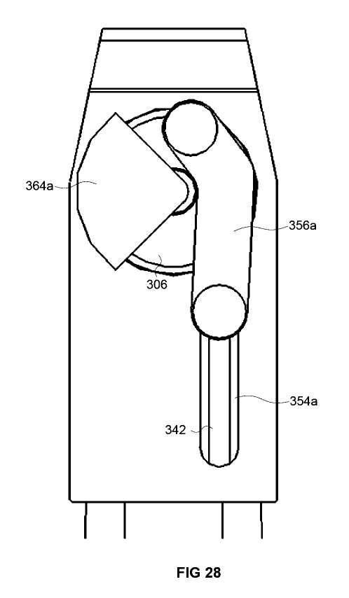

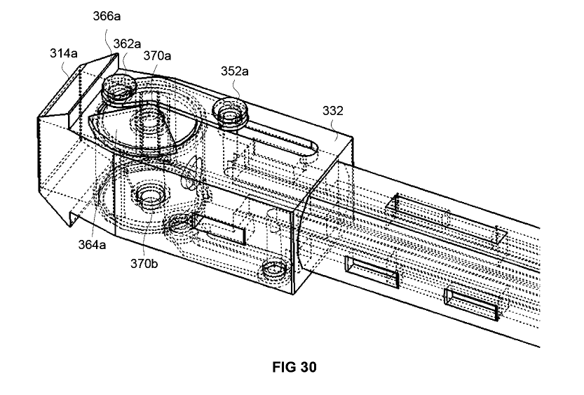

FIGS. 25-30 show additional views of the device of FIG. 24 some of which are plan views, some of which are perspective views and some of which show hidden lines keeping elements that cannot be directly seen.

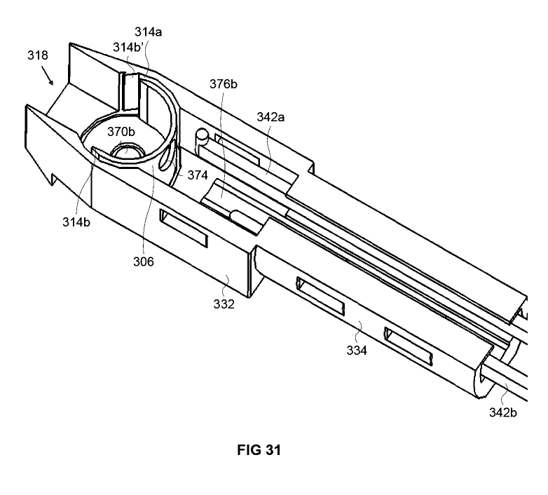

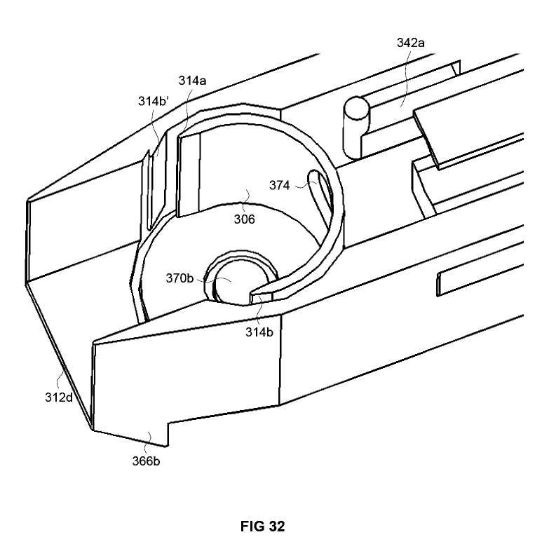

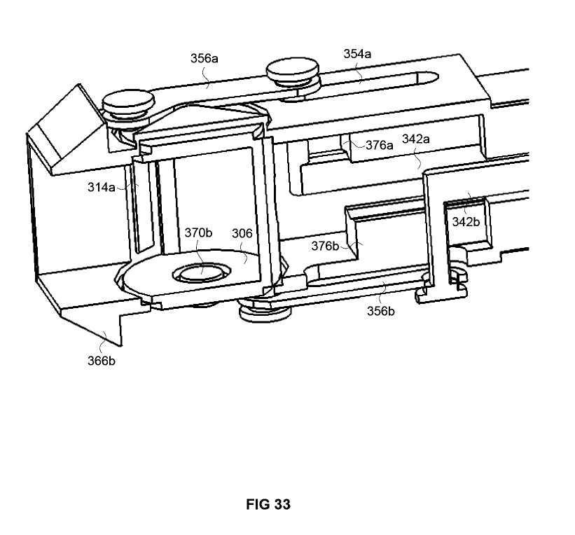

FIGS. 31 and 32 provide cross-sectional views through the device of FIGS. 24-30 along a plane that is perpendicular to a preferred build axis while FIG. 33 provides a cross-sectional view through the device of FIGS. 24-32 along a plane that includes the build axis and the longitudinal axis of the device.

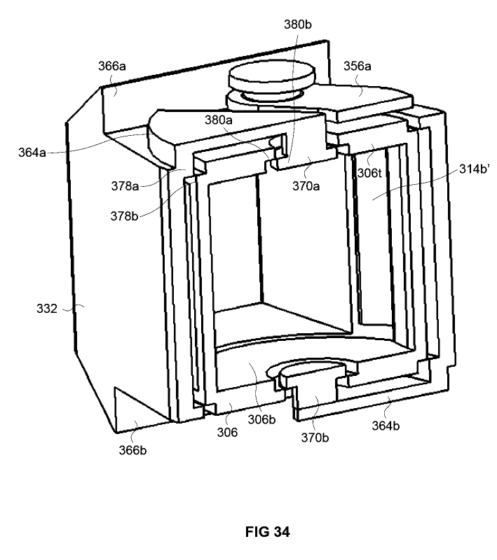

FIG. 34 shows a cross-sectional view of the distal end of the device of FIGS. 24-33 as cut through the barrel pivots.

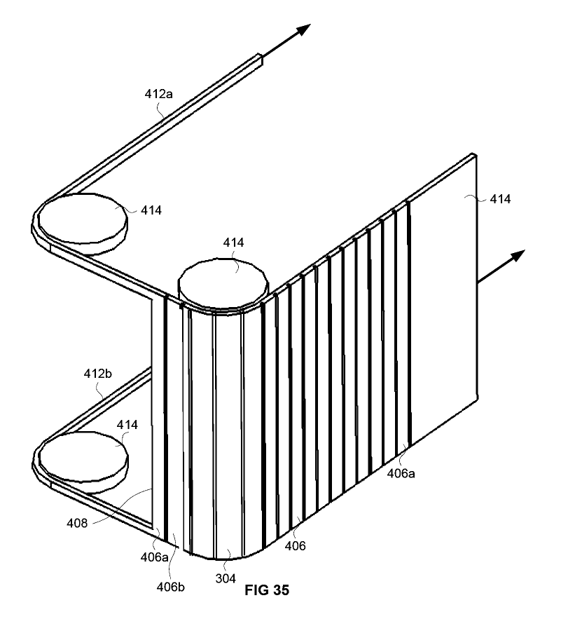

FIG. 35 shows a partial view of a distal end of a device according to a fourth embodiment of the invention.

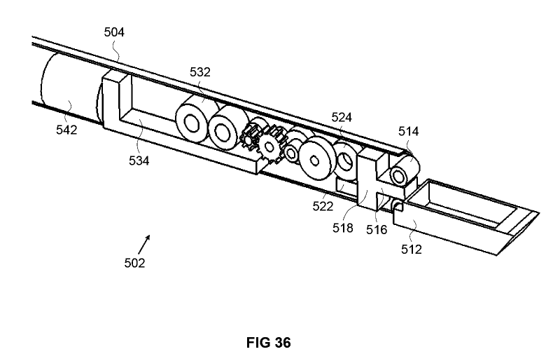

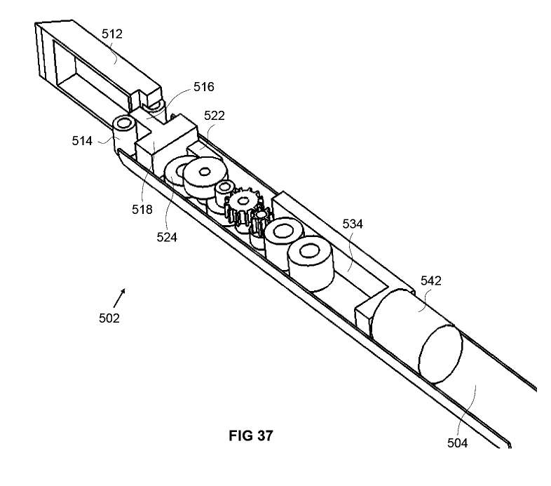

FIGS. 36-37 show a partial view of a device according to a fifth embodiment of the invention.

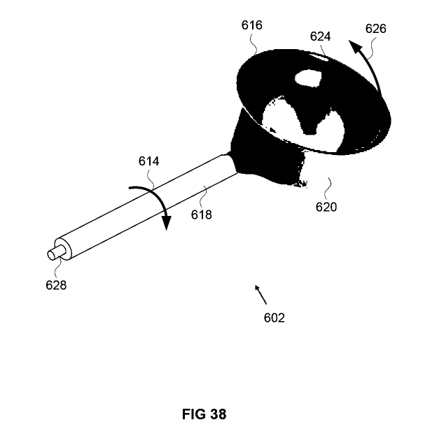

FIG. 38 shows a device according to a sixth embodiment of the invention.

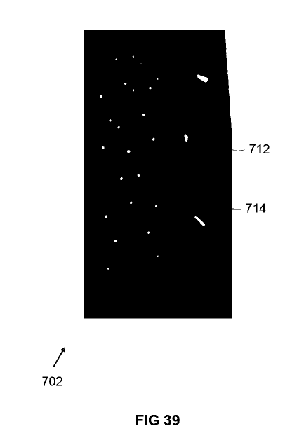

FIG. 39 depicts a device according to a seventh embodiment of the invention.

### DETAILED DESCRIPTION OF PREFERRED EMBODIMENTS:

### Electrochemical Fabrication In Preferred Embodiments

FIGS. 1A-1G, 2A-2F, and 3A-3C illustrate various features of one form of electrochemical fabrication. Other electrochemical fabrication techniques are set forth in the '630 patent referenced above, in the various previously incorporated publications, in various other patents and patent applications incorporated herein by reference. Still others may be derived from combinations of various approaches described in these publications, patents, and applications, or are otherwise known or ascertainable by those of skill in the art from the teachings set forth herein. All of these techniques may be combined with those of the various embodiments of various aspects of the invention to yield enhanced embodiments. Still other embodiments may be derived from combinations of the various embodiments explicitly set forth herein.

FIGS. 4A-4I illustrate various stages in the formation of a single layer of a multi-layer fabrication process where a second metal is deposited on a first metal as well as in openings in the first metal so that the first and second metal form part of the layer. In FIG. 4A a side view of a substrate 82 is shown, onto which patternable photoresist 84 is cast as shown in FIG. 4B. In FIG. 4C, a pattern of resist is shown that results from the curing, exposing, and developing of the resist. The patterning of the photoresist 84 results in openings or apertures 92(a)-92(c) extending from a surface 86 of the photoresist through the thickness of the photoresist to surface 88 of the substrate 82. In FIG. 4D a metal 94 (e.g. nickel) is shown as having been electroplated into the openings 92(a)-92(c). In FIG. 4E the photoresist has been removed (i.e. chemically stripped) from the substrate to expose regions of the substrate 82 which are not covered with the first metal 94. In FIG. 4F a second metal 96 (e.g. silver) is shown as having been blanket electroplated over the entire exposed portions of the substrate 82 (which is conductive) and over the first metal 94 (which is also conductive). FIG. 4G depicts the completed first layer of the structure which has resulted from the planarization of the first and second metals down to a height that exposes the first metal and sets a thickness for the first layer. In FIG. 4H the result of repeating the process steps shown in FIGS. 4B-4G several times to form a multi-layer structure are shown where each layer consists of two materials. For most applications, one of these materials is removed as shown in FIG. 4I to yield a desired 3-D structure 98 (e.g. component or device).

Various embodiments of various aspects of the invention are directed to formation of three-dimensional structures from materials some of which may be electrodeposited or electroless deposited. Some of these structures may be formed form a single build level formed from one or more deposited materials while others are formed from a plurality of build layers each including at least two materials (e.g. two or more layers, more preferably five or more layers, and most preferably ten or more layers). In some embodiments, layer thicknesses may be as small as one micron or as large as fifty microns. In other embodiments, thinner layers may be used while in other embodiments, thicker layers may be used. In some embodiments structures having features positioned with micron level precision and minimum features size on the order of tens of microns are to be formed. In other embodiments structures with less precise feature placement and/or larger minimum features may be formed. In still other embodiments, higher precision and smaller minimum feature sizes may be desirable.

The various embodiments, alternatives, and techniques disclosed herein may form multi-layer structures using a single patterning technique on all layers or using different patterning techniques on different layers. For example, Various embodiments of the invention may perform selective patterning operations using conformable contact masks and masking operations (i.e. operations that use masks which are contacted to but not adhered to a substrate), proximity masks and masking operations (i.e. operations that use masks that at least partially selectively shield a substrate by their proximity to the substrate even if contact is not made), non-conformable masks and masking operations (i.e. masks and operations based on masks whose contact surfaces are not significantly conformable), and/or adhered masks and masking operations (masks and operations that use masks that are adhered to a substrate onto which selective deposition or etching is to occur as opposed to only being contacted to it). Conformable contact masks, proximity masks, and non-conformable contact masks share the property that they are preformed and brought to, or in proximity to, a surface which is to be treated (i.e. the exposed portions of the surface are to be treated). These masks can generally be removed without damaging the mask or the surface that received treatment to which they were contacted, or located in proximity to. Adhered masks are generally formed on the surface to be treated (i.e. the portion of that surface that is to be masked) and bonded to that surface such that they cannot be separated from that surface without being completely destroyed damaged beyond any point of reuse. Adhered masks may be formed in a number of ways including (1) by application of a photoresist, selective exposure of the photoresist, and then development of the photoresist, (2) selective transfer of pre-patterned masking material, and/or (3) direct formation of masks from computer controlled depositions of material.

Patterning operations may be used in selectively depositing material and/or may be used in the selective etching of material. Selectively etched regions may be selectively filled in or filled in via blanket deposition, or the like, with a different desired material. In some embodiments, the layer-by-layer build up may involve the simultaneous formation of portions of multiple layers. In some embodiments, depositions made in association with some layer levels may result in depositions to regions associated with other layer levels (i.e. regions that lie within the top and bottom boundary levels that define a different layer's geometric configuration). Such use of selective etching and interlaced material deposition in association with multiple layers is described in U.S. patent application Ser. No. 10/434,519, by Smalley, and entitled “Methods of and Apparatus for Electrochemically Fabricating Structures Via Interlaced Layers or Via Selective Etching and Filling of Voids layer elements” which is hereby incorporated herein by reference as if set forth in full.

Temporary substrates on which structures may be formed may be of the sacrificial-type (i.e. destroyed or damaged during separation of deposited materials to the extent they can not be reused), non-sacrificial-type (i.e. not destroyed or excessively damaged, i.e. not damaged to the extent they may not be reused, e.g. with a sacrificial or release layer located between the substrate and the initial layers of a structure that is formed). Non-sacrificial substrates may be considered reusable, with little or no rework (e.g. replanarizing one or more selected surfaces or applying a release layer, and the like) though they may or may not be reused for a variety of reasons.

### DEFINITIONS

This section of the specification is intended to set forth definitions for a number of specific terms that may be useful in describing the subject matter of the various embodiments of the invention. It is believed that the meanings of most if not all of these terms is clear from their general use in the specification but they are set forth hereinafter to remove any ambiguity that may exist. It is intended that these definitions be used in understanding the scope and limits of any claims that use these specific terms. As far as interpretation of the claims of this patent disclosure are concerned, it is intended that these definitions take presence over any contradictory definitions or allusions found in any materials which are incorporated herein by reference.

“Build” as used herein refers, as a verb, to the process of building a desired structure or plurality of structures from a plurality of applied or deposited materials which are stacked and adhered upon application or deposition or, as a noun, to the physical structure or structures formed from such a process. Depending on the context in which the term is used, such physical structures may include a desired structure embedded within a sacrificial material or may include only desired physical structures which may be separated from one another or may require dicing and/or slicing to cause separation.

“Build axis” or “build orientation” is the axis or orientation that is substantially perpendicular to substantially planar levels of deposited or applied materials that are used in building up a structure. The planar levels of deposited or applied materials may be or may not be completely planar but are substantially so in that the overall extent of their cross-sectional dimensions are significantly greater than the height of any individual deposit or application of material (e.g. 100, 500, 1000, 5000, or more times greater). The planar nature of the deposited or applied materials may come about from use of a process that leads to planar deposits or it may result from a planarization process (e.g. a process that includes mechanical abrasion, e.g. lapping, fly cutting, grinding, or the like) that is used to remove material regions of excess height. Unless explicitly noted otherwise, “vertical” as used herein refers to the build axis or nominal build axis (if the layers are not stacking with perfect registration) while “horizontal” refers to a direction within the plane of the layers (i.e. the plane that is substantially perpendicular to the build axis).

“Build layer” or “layer of structure” as used herein does not refer to a deposit of a specific material but instead refers to a region of a build located between a lower boundary level and an upper boundary level which generally defines a single cross-section of a structure being formed or structures which are being formed in parallel. Depending on the details of the actual process used to form the structure, build layers are generally formed on and adhered to previously formed build layers. In some processes the boundaries between build layers are defined by planarization operations which result in successive build layers being formed on substantially planar upper surfaces of previously formed build layers. In some embodiments, the substantially planar upper surface of the preceding build layer may be textured to improve adhesion between the layers. In other build processes, openings may exist in or be formed in the upper surface of a previous but only partially formed build layers such that the openings in the previous build layers are filled with materials deposited in association with current build layers which will cause interlacing of build layers and material deposits. Such interlacing is described in U.S. patent application Ser. No. 10/434,519. This referenced application is incorporated herein by reference as if set forth in full. In most embodiments, a build layer includes at least one primary structural material and at least one primary sacrificial material. However, in some embodiments, two or more primary structural materials may used without a primary sacrificial material (e.g. when one primary structural material is a dielectric and the other is a conductive material). In some embodiments, build layers are distinguishable from each other by the source of the data that is used to yield patterns of the deposits, applications, and/or etchings of material that form the respective build layers. For example, data descriptive of a structure to be formed which is derived from data extracted from different vertical levels of a data representation of the structure define different build layers of the structure. The vertical separation of successive pairs of such descriptive data may define the thickness of build layers associated with the data. As used herein, at times, “build layer” may be loosely referred simply as “layer”. In many embodiments, deposition thickness of primary structural or sacrificial materials (i.e. the thickness of any particular material after it is deposited) is generally greater than the layer thickness and a net deposit thickness is set via one or more planarization processes which may include, for example, mechanical abrasion (e.g. lapping, fly cutting, polishing, and the like) and/or chemical etching (e.g. using selective or non-selective etchants). The lower boundary and upper boundary for a build layer may be set and defined in different ways. From a design point of view they may be set based on a desired vertical resolution of the structure (which may vary with height). From a data manipulation point of view, the vertical layer boundaries may be defined as the vertical levels at which data descriptive of the structure is processed or the layer thickness may be defined as the height separating successive levels of cross-sectional data that dictate how the structure will be formed. From a fabrication point of view, depending on the exact fabrication process used, the upper and lower layer boundaries may be defined in a variety of different ways. For example by planarization levels or effective planarization levels (e.g. lapping levels, fly cutting levels, chemical mechanical polishing levels, mechanical polishing levels, vertical positions of structural and/or sacrificial materials after relatively uniform etch back following a mechanical or chemical mechanical planarization process). For example, by levels at which process steps or operations are repeated. At levels at which, at least theoretically, lateral extends of structural material can be changed to define new cross-sectional features of a structure.

“Layer thickness” is the height along the build axis between a lower boundary of a build layer and an upper boundary of that build layer.

“Planarization” is a process that tends to remove materials, above a desired plane, in a substantially non-selective manner such that all deposited materials are brought to a substantially common height or desired level (e.g. within 20%, 10%, 5%, or even 1% of a desired layer boundary level). For example, lapping removes material in a substantially non-selective manner though some amount of recession one material or another may occur (e.g. copper may recess relative to nickel). Planarization may occur primarily via mechanical means, e.g. lapping, grinding, fly cutting, milling, sanding, abrasive polishing, frictionally induced melting, other machining operations, or the like (i.e. mechanical planarization). Mechanical planarization maybe followed or proceeded by thermally induced planarization (.e.g. melting) or chemically induced planarization (e.g. etching). Planarization may occur primarily via a chemical and/or electrical means (e.g. chemical etching, electrochemical etching, or the like). Planarization may occur via a simultaneous combination of mechanical and chemical etching (e.g. chemical mechanical polishing (CMP)).

“Structural material” as used herein refers to a material that remains part of the structure when put into use.

“Supplemental structural material” as used herein refers to a material that forms part of the structure when the structure is put to use but is not added as part of the build layers but instead is added to a plurality of layers simultaneously (e.g. via one or more coating operations that applies the material, selectively or in a blanket fashion, to a one or more surfaces of a desired build structure that has been released from a sacrificial material.

“Primary structural material” as used herein is a structural material that forms part of a given build layer and which is typically deposited or applied during the formation of that build layer and which makes up more than 20% of the structural material volume of the given build layer. In some embodiments, the primary structural material may be the same on each of a plurality of build layers or it may be different on different build layers. In some embodiments, a given primary structural material may be formed from two or more materials by the alloying or diffusion of two or more materials to form a single material.

“Secondary structural material” as used herein is a structural material that forms part of a given build layer and is typically deposited or applied during the formation of the given build layer but is not a primary structural material as it individually accounts for only a small volume of the structural material associated with the given layer. A secondary structural material will account for less than 20% of the volume of the structural material associated with the given layer. In some preferred embodiments, each secondary structural material may account for less than 10%, 5%, or even 2% of the volume of the structural material associated with the given layer. Examples of secondary structural materials may include seed layer materials, adhesion layer materials, barrier layer materials (e.g. diffusion barrier material), and the like. These secondary structural materials are typically applied to form coatings having thicknesses less than 2 microns, 1 micron, 0.5 microns, or even 0.2 microns). The coatings may be applied in a conformal or directional manner (e.g. via CVD, PVD, electroless deposition, or the like). Such coatings may be applied in a blanket manner or in a selective manner. Such coatings may be applied in a planar manner (e.g. over previously planarized layers of material) as taught in U.S. patent application Ser. No. 10/607,931. In other embodiments, such coatings may be applied in a non-planar manner, for example, in openings in and over a patterned masking material that has been applied to previously planarized layers of material as taught in U.S. patent application Ser. No. 10/841,383. These referenced applications are incorporated herein by reference as if set forth in full herein.

“Functional structural material” as used herein is a structural material that would have been removed as a sacrificial material but for its actual or effective encapsulation by other structural materials. Effective encapsulation refers, for example, to the inability of an etchant to attack the functional structural material due to inaccessibility that results from a very small area of exposure and/or due to an elongated or tortuous exposure path. For example, large (10,000 μm²) but thin (e.g. less than 0.5 microns) regions of sacrificial copper sandwiched between deposits of nickel may define regions of functional structural material depending on ability of a release etchant to remove the sandwiched copper.

“Sacrificial material” is material that forms part of a build layer but is not a structural material. Sacrificial material on a given build layer is separated from structural material on that build layer after formation of that build layer is completed and more generally is removed from a plurality of layers after completion of the formation of the plurality of layers during a “release” process that removes the bulk of the sacrificial material or materials. In general sacrificial material is located on a build layer during the formation of one, two, or more subsequent build layers and is thereafter removed in a manner that does not lead to a planarized surface. Materials that are applied primarily for masking purposes, i.e. to allow subsequent selective deposition or etching of a material, e.g. photoresist that is used in forming a build layer but does not form part of the build layer) or that exist as part of a build for less than one or two complete build layer formation cycles are not considered sacrificial materials as the term is used herein but instead shall be referred as masking materials or as temporary materials. These separation processes are sometimes referred to as a release process and may or may not involve the separation of structural material from a build substrate. In many embodiments, sacrificial material within a given build layer is not removed until all build layers making up the three-dimensional structure have been formed. Of course sacrificial material may be, and typically is, removed from above the upper level of a current build layer during planarization operations during the formation of the current build layer. Sacrificial material is typically removed via a chemical etching operation but in some embodiments may be removed via a melting operation or electrochemical etching operation. In typical structures, the removal of the sacrificial material (i.e. release of the structural material from the sacrificial material) does not result in planarized surfaces but instead results in surfaces that are dictated by the boundaries of structural materials located on each build layer. Sacrificial materials are typically distinct from structural materials by having different properties therefrom (e.g. chemical etchability, hardness, melting point, etc.) but in some cases, as noted previously, what would have been a sacrificial material may become a structural material by its actual or effective encapsulation by other structural materials. Similarly, structural materials may be used to form sacrificial structures that are separated from a desired structure during a release process via the sacrificial structures being only attached to sacrificial material or potentially by dissolution of the sacrificial structures themselves using a process that is insufficient to reach structural material that is intended to form part of a desired structure. It should be understood that in some embodiments, small amounts of structural material may be removed, after or during release of sacrificial material. Such small amounts of structural material may have been inadvertently formed due to imperfections in the fabrication process or may result from the proper application of the process but may result in features that are less than optimal (e.g. layers with stairs steps in regions where smooth sloped surfaces are desired. In such cases the volume of structural material removed is typically minuscule compared to the amount that is retained and thus such removal is ignored when labeling materials as sacrificial or structural. Sacrificial materials are typically removed by a dissolution process, or the like, that destroys the geometric configuration of the sacrificial material as it existed on the build layers. In many embodiments, the sacrificial material is a conductive material such as a metal. As will be discussed hereafter, masking materials though typically sacrificial in nature are not termed sacrificial materials herein unless they meet the required definition of sacrificial material.

“Supplemental sacrificial material” as used herein refers to a material that does not form part of the structure when the structure is put to use and is not added as part of the build layers but instead is added to a plurality of layers simultaneously (e.g. via one or more coating operations that applies the material, selectively or in a blanket fashion, to a one or more surfaces of a desired build structure that has been released from an initial sacrificial material. This supplemental sacrificial material will remain in place for a period of time and/or during the performance of certain post layer formation operations, e.g. to protect the structure that was released from a primary sacrificial material, but will be removed prior to putting the structure to use.

“Primary sacrificial material” as used herein is a sacrificial material that is located on a given build layer and which is typically deposited or applied during the formation of that build layer and which makes up more than 20% of the sacrificial material volume of the given build layer. In some embodiments, the primary sacrificial material may be the same on each of a plurality of build layers or may be different on different build layers. In some embodiments, a given primary sacrificial material may be formed from two or more materials by the alloying or diffusion of two or more materials to form a single material.

“Secondary sacrificial material” as used herein is a sacrificial material that is located on a given build layer and is typically deposited or applied during the formation of the build layer but is not a primary sacrificial materials as it individually accounts for only a small volume of the sacrificial material associated with the given layer. A secondary sacrificial material will account for less than 20% of the volume of the sacrificial material associated with the given layer. In some preferred embodiments, each secondary sacrificial material may account for less than 10%, 5%, or even 2% of the volume of the sacrificial material associated with the given layer. Examples of secondary structural materials may include seed layer materials, adhesion layer materials, barrier layer materials (e.g. diffusion barrier material), and the like. These secondary sacrificial materials are typically applied to form coatings having thicknesses less than 2 microns, 1 micron, 0.5 microns, or even 0.2 microns). The coatings may be applied in a conformal or directional manner (e.g. via CVD, PVD, electroless deposition, or the like). Such coatings may be applied in a blanket manner or in a selective manner. Such coatings may be applied in a planar manner (e.g. over previously planarized layers of material) as taught in U.S. patent application Ser. No. 10/607,931. In other embodiments, such coatings may be applied in a non-planar manner, for example, in openings in and over a patterned masking material that has been applied to previously planarized layers of material as taught in U.S. patent application Ser. No. 10/841,383. These referenced applications are incorporated herein by reference as if set forth in full herein.

“Adhesion layer”, “seed layer”, “barrier layer”, and the like refer to coatings of material that are thin in comparison to the layer thickness and thus generally form secondary structural material portions or sacrificial material portions of some layers. Such coatings may be applied uniformly over a previously formed build layer, they may be applied over a portion of a previously formed build layer and over patterned structural or sacrificial material existing on a current (i.e. partially formed) build layer so that a non-planar seed layer results, or they may be selectively applied to only certain locations on a previously formed build layer. In the event such coatings are non-selectively applied, selected portions may be removed (1) prior to depositing either a sacrificial material or structural material as part of a current layer or (2) prior to beginning formation of the next layer or they may remain in place through the layer build up process and then etched away after formation of a plurality of build layers.

“Masking material” is a material that may be used as a tool in the process of forming a build layer but does not form part of that build layer. Masking material is typically a photopolymer or photoresist material or other material that may be readily patterned. Masking material is typically a dielectric. Masking material, though typically sacrificial in nature, is not a sacrificial material as the term is used herein. Masking material is typically applied to a surface during the formation of a build layer for the purpose of allowing selective deposition, etching, or other treatment and is removed either during the process of forming that build layer or immediately after the formation of that build layer.

“Multilayer structures” are structures formed from multiple build layers of deposited or applied materials.

“Multilayer three-dimensional (or 3D or 3-D) structures” are Multilayer Structures that meet at least one of two criteria: (1) the structural material portion of at least two layers of which one has structural material portions that do not overlap structural material portions of the other.

“Complex multilayer three-dimensional (or 3D or 3-D) structures” are multilayer three-dimensional structures formed from at least three layers where a line may be defined that hypothetically extends vertically through at least some portion of the build layers of the structure will extend from structural material through sacrificial material and back through structural material or will extend from sacrificial material through structural material and back through sacrificial material (these might be termed vertically complex multilayer three-dimensional structures). Alternatively, complex multilayer three-dimensional structures may be defined as multilayer three-dimensional structures formed from at least two layers where a line may be defined that hypothetically extends horizontally through at least some portion of a build layer of the structure that will extend from structural material through sacrificial material and back through structural material or will extend from sacrificial material through structural material and back through sacrificial material (these might be termed horizontally complex multilayer three-dimensional structures). Worded another way, in complex multilayer three-dimensional structures, a vertically or horizontally extending hypothetical line will extend from one or structural material or void (when the sacrificial material is removed) to the other of void or structural material and then back to structural material or void as the line is traversed along at least a portion of the line.

“Moderately complex multilayer three-dimensional (or 3D or 3-D) structures are complex multilayer 3D structures for which the alternating of void and structure or structure and void not only exists along one of a vertically or horizontally extending line but along lines extending both vertically and horizontally.

“Highly complex multilayer (or 3D or 3-D) structures are complex multilayer 3D structures for which the structure-to-void-to-structure or void-to-structure-to-void alternating occurs once along the line but occurs a plurality of times along a definable horizontally or vertically extending line.

“Up-facing feature” is an element dictated by the cross-sectional data for a given build layer “n” and a next build layer “n+1” that is to be formed from a given material that exists on the build layer “n” but does not exist on the immediately succeeding build layer “n+1”. For convenience the term “up-facing feature” will apply to such features regardless of the build orientation.

“Down-facing feature” is an element dictated by the cross-sectional data for a given build layer “n” and a preceding build layer “n−1” that is to be formed from a given material that exists on build layer “n” but does not exist on the immediately preceding build layer “n−1”. As with up-facing features, the term “down-facing feature” shall apply to such features regardless of the actual build orientation.

“Continuing region” is the portion of a given build layer “n” that is dictated by the cross-sectional data for the given build layer “n” , a next build layer “n+1” and a preceding build layer “n−1” that is neither up-facing nor down-facing for the build layer “n”.

“Minimum feature size” refers to a necessary or desirable spacing between structural material elements on a given layer that are to remain distinct in the final device configuration. If the minimum feature size is not maintained on a given layer, the fabrication process may result in structural material inadvertently bridging the two structural elements due to masking material failure or failure to appropriately fill voids with sacrificial material during formation of the given layer such that during formation of a subsequent layer structural material inadvertently fills the void. More care during fabrication can lead to a reduction in minimum feature size or a willingness to accept greater losses in productivity can result in a decrease in the minimum feature size. However, during fabrication for a given set of process parameters, inspection diligence, and yield (successful level of production) a minimum design feature size is set in one way or another. The above described minimum feature size may more appropriately be termed minimum feature size of sacrificial material regions. Conversely a minimum feature size for structure material regions (minimum width or length of structural material elements) may be specified. Depending on the fabrication method and order of deposition of structural material and sacrificial material, the two types of minimum feature sizes may be different. In practice, for example, using electrochemical fabrication methods and described herein, the minimum features size on a given layer may be roughly set to a value that approximates the layer thickness used to form the layer and it may be considered the same for both structural and sacrificial material widths and lengths. In some more rigorously implemented processes, examination regiments, and rework requirements, it may be set to an amount that is 80%, 50%, or even 30% of the layer thickness. Other values or methods of setting minimum feature sizes may be set.

Biopsy Devices

Various embodiments of the invention relate to devices for removal of small volumes of tissue from the body. Such removal is a required part of biopsy procedures of the image-guided or non image-guided type. The devices of these embodiments are applicable to performance of biopsies on many types of tissue and organs, particularly soft tissue, including: intestines, stomach, breast, pancreas, lung, liver, brain, skin, prostate, thyroid, heart, and other organs and tissues.

Prior devices for such removal purposes are described, for example, in the paper “Barbed micro-spikes for micro-scale biopsy” (Byun et. Al, J. Micromech. Microeng. 15 (2005) 1279-1284). This referenced paper is incorporated herein by reference. In this paper, a barbed biopsy device is fabricated from silicon. It is pushed into the tissue to be biopsied and then withdrawn, such that barbs formed on the sides of protruding sections of the device retain tissue. It is noted by the authors that the ability of such devices to excise tissue is greatly enhanced by the addition of the barbs. The device described in this paper has significant disadvantages and limitations. These limitations include:
Silicon is a brittle material, so the risk of device breakage and harm to the patient is significant.
Since there is no ‘floor’ and ‘ceiling’ surrounding the barbs to retain the specimen, it can easily slip away from the barbs and be lost. Loss of the specimen may in some cases (e.g., the cardiovascular system) present a significant risk to the patient. Moreover, the lack of a floor and ceiling makes the sample that can be obtained typically smaller, and the ability to obtain it less reliable, since tissue can ‘escape capture’ through the open top and bottom.

In preferred embodiments of the invention, the devices are preferably made from metal (e.g., nickel-cobalt, nickel-titanium, stainless steel, nickel-phosphorous) and are preferably produced using a multi-layer micro-manufacturing process such an electrochemical fabrication process described herein above. In other embodiments, other materials may be used or incorporated into the devices and other fabrication processes may be used.

### First Preferred Embodiment

FIG. 5 provides a perspective overview of the device of a first preferred embodiment. The device 102 includes a body portion 112 terminating at the distal end 126 in two jaws 114a and 114b that are initially substantially parallel, separated by a slot 116. The distal ends of the jaws 114a and 114b are preferably made sharp to serve as cutting edges 124a and 124b and are separated by an inlet 118. An aperture 122 is provided in the body 112 of the device to provide clearance for squeezing the device, as will be discussed below. In some alternative embodiments, such an aperture may not be necessary. The aperture 122 also serves, as do a plurality of release holes 132, to allow sacrificial material to be etched out (e.g. after fabrication of all layers of the device) when the device is fabricated using an electrochemical fabrication process that involves the use of a sacrificial material to fill portions of each layer that are not occupied by a structural material that defines part of the device. Also shown in the figure is a handle 134, at the proximal end 128 of the body 112, which controls whether the jaws remain closed for sampling or whether they are opened for specimen removal. The body preferably as a width, W, and a height, H, are selected to produce samples of a desired size while minimizing damage to tissue that must be penetrated to get to the extraction site. The width and height may also be selected to allow the device to fit through a lumen in a catheter or other delivery device if it is to be passed to the extraction site via such a passage.

FIGS. 6-7 provide alternative, perspective views of the distal end 126 of the device of FIG. 5 so that barbs 142a and 142b lining the sides of the left and right jaws, respectively. In use, these barbs are oriented to allow tissue to slide into the inlet but tend to retain or capture the tissue that has entered the inlet (i.e. limit the ability of tissue to slide back out of the inlet).

FIG. 8A and 8B illustrate schematically a closed and open state of the jaws of the device of FIG. 5. In FIG. 8A the jaws 114a and 114b are in a substantially parallel configuration (i.e. not opened). This is the state of the jaws when the device 102 is inserted into tissue to obtain a specimen. In FIG. 8B, the jaws 114a and 114b have been forced into an open configuration which allows easy removal of the organ or tissue specimen. This open, V-shaped, or spread configuration results from squeezing the device in the region of the aperture 122 which results in the deforming of the body of the device (elastically or plastically) and the spreading open of the jaws 114a and 114b about fulcrum which results in the deforming of the body of the device (elastically or plastically). In some alternative embodiments, the fulcrum may take on other configurations so as to enhance the ability of the jaws to open. For example, the fulcrum may be physically bonded, adhered, or connected to only one jaw or the other so that upon squeezing the aperture, one jaw tends to undergo a larger angular displacement than the other. In still other alternatives the fulcrum may take on a sharper or more pointed configuration where it contacts or both jaws. In still other embodiments, the fulcrum may be replaced by a pin and rings connected separately to one or both jaws to allow pivoting or rotation from a closed to an open state.

In this first embodiment, the device is provided with a mechanism to prevent premature spreading of the jaws that may result from improper handling, tissue reaction forces, and the like. In alternative embodiments, such a mechanism may not be necessary. An example of such a mechanism or retainer 152 is visible in the cross-sectional top view of the device as shown in FIG. 9. The handle 134 is attached to a retainer shaft 154 that passes through an opening 156 that extends along the length of the body 112 of the device 102. At the distal end of this shaft a retainer 152 is located which preferably has a tapered shape such that when the shaft is moved toward the distal end of the body, the distal end of the retainer 152 can engage bosses 158a and 158b on the jaws to prevent the jaws from spreading open. The bosses may be located at the proximal end of the jaws or they may, as in the present case, be located between the distal and proximal ends of the jaws. In some alternative devices, the retainer may be located in the region of the aperture and simply prevent collapse of the aperture and thus prevent the spreading of the jaws. In some alternative embodiments, as noted above, a mechanism to inhibit spreading may not be required.

In some embodiments, as in the present embodiment, the barbs on the jaws may have outward facing sloped surfaces that allow easy slide in entry of tissue while their back surfaces may be substantially perpendicular to the slide-in direction. In still other embodiments the slope of the back ends of the barbs may not be perpendicular to the slide-in direction but may be more steeply sloped than the outward facing surfaces. In still other embodiments, the back ends of the barbs may be undercut (i.e. oblique relative to the slide-in direction) to provide a positive retention forces (i.e. a true barb) that inhibits tissue from being released from the distal end of the device.

In some alternative embodiments, the jaws may not form part of the body of the device but instead may be located at a distal end of a shaft that is insertable into the body of the device (e.g. a device within a device). The shaft and jaws may be insertable and extractable from the body of the device while the body of the device remains at a substantially fixed location within the body of the patient. In some alternative embodiments, the specimen may be retrieved by pulling the jaws back to the location of an aperture within the body of the device and then removing the specimen through the aperture either with the aid of the jaws spreading open or without the jaws being transformed to an open position. In some embodiments, the specimen may be extracted from the proximal end of the device or it may be extracted once the entire device is removed from the body of the patient.

FIGS. 10-11 provide respectively a plan view of the distal end of the device of FIG. 5 holding a specimen and a perspective view of the distal end of the device. FIG. 10 depicts how a specimen that enters the inlet of the device is retained in the chamber formed by the jaws by virtue of the proximally-pointing barbs. In the views of FIGS. 10 and 11, the retainer 152 is in a proximal position, as opposed to its engaged distal position, thus showing the jaw bosses 158a and 158b when not engaged with the retainer 152 and thus showing the state of the device when the jaws 114a and 114b are capable of being opened. When in the depicted closed position the jaws may be considered to form an inlet or extraction chamber that has an open end and a narrow slot 156 on its top and bottom surfaces. In some embodiments, barbs may be provided not only on the left and right sides of the jaws but also on the top and bottom surfaces of each jaw. (i.e. on the floor and ceiling of the inlet or extraction chamber), such that the entire chamber is lined with barbs.

FIG. 12 provides a cross-sectional view of the device of FIG. 5 in the region of the fulcrum. The fulcrum 144 is located between the slot that separates the jaws 114a and 114b and the aperture 122. As depicted, the fulcrum 144 is a region of the body 112 of the device that resists crushing yet has an aperture 162 through which the retainer shaft can pass. As in the present embodiment, it may be advantageous for retainer 152 to be too large to allow it to pass through aperture 162 as in some embodiments the retainer may need only travel a short distance from an engaged to an unengaged position to allow locking and unlocking of the jars. In other embodiments, the retainer 152 may be small enough to be extracted from the body of the device. Such a retraction maybe possible, for example, by simply pulling the retainer 152 backward or by first twisting the retainer 152 to a selected orientation that aligns portions of the retainer 152 with portions of the aperture 162 which is then followed by extraction.

The device of FIG. 5 may be operated according to the following sequence of operations:
1. The handle is moved distally relative to the body 112 of the device 102 so that the retainer engages the bosses on the jaws.
2. While the handle is held in this distal position, relative to the body of the device, the device is pushed into the tissue (e.g., by pushing distally on the handle or distally along the body of the device) to be sampled, causing a specimen of tissue to enter the device inlet while its edges are being cut by edges 124a and 124b. Since the jaws are prevented from spreading by the retainer engaging the bosses, the specimen is slightly compressed within the inlet of the device.
3. The device 102 and sample are then withdrawn from the tissue. Since the specimen within the barbed jaws is largely unable to be removed from the distal ends of the jaws, the specimen becomes separated adjacent body the tissue. This extraction may occur by pulling the body of the device in the proximal direction or possibly by pulling the handle the proximal direction. If the handle is pulled, the jaws may still remain closed.
4. Once the device 102 is removed from the patient, if not already moved, the handle is moved proximally, enabling spreading of the jaws.
5. The body of the device 102 is squeezed (e.g., by forceps) on either side of the aperture, causing the jaws to open. At this time, the specimen either falls out onto a suitable receiver or may readily be removed (e.g., using a sharp instrument).

In some alternative embodiments, the retention mechanism may serve an additional function. Upon normal pushing of the handle relative to the body of the device, the retention mechanism may simply keep the jars from opening. Upon harder pushing, the retention device may actually cause the jars to come closer together thus causing a tighter griping of the sample and stronger retention of the sample, particularly during separation of the sample from other body tissue. In still other alternative embodiments, a squeezing of the jars together may occur via other mechanisms (including mechanisms actuated upon pulling instead of pushing).

If desired, a catch, detent, or other mechanism can be provided to keep the retainer engaging the bosses while the device is inserted into tissue. The device may be made small in size, e.g., with a width W (see FIG. 5) on the order of 1 mm or smaller and a height H on the same order or even smaller. Other mechanical mechanisms and operations may be used to ensure that the bosses remain in engaged during insertion and, if necessary, during extraction as well.

Preferred methods of producing the devices of various embodiments of the invention use an electrochemical fabrication process such as the EFAB® process of Microfabrica Corporation of Van Nuys, Calif.). In such production processes, the body of the device and movable parts are preferably formed in simultaneously and in their respective positions. For example, to allow required minimum features sizes to be maintained during formation, the retainer and the bosses would preferably be formed while in an unengaged position with sacrificial material separating them. In some fabrication embodiments the device would be oriented so that the build axis is parallel to the height of the device while in other embodiments, the build axis might be parallel to the width of the device. In some embodiments the retainer shaft may be formed with wider portions and narrower portion so that it may be appropriately spaced from internal portions of the body 112 during fabrication (e.g. so that minimum feature sizes are maintained).

Devices like that of FIG. 5 and similar devices may be fabricated by other than electrochemical fabrication methods. An example of such an alternative method is shown in FIGS. 13A-13I, in which a device is fabricated from a metal tube. In alternative embodiments, the sequence of operations may be varied from that depicted in FIGS. 13A-13I.

In FIG. 13A, a tube 182 with a sharpened distal end 184 is provided. In the figures, the distal end of the tube is on the right while the proximal end is on the left. The tube may be sharpened, e.g., using grinding, electrochemical sharpening, or the like.

In FIG. 13B the tube 182 is shown after cuts 186 have been made into its sides (e.g., using a slitting saw, electrical discharge machining (i.e., EDM), or the like.

In FIG. 13C, the tube is depicted after apertures 190 have been cut on the top and bottom surfaces of the tube.

In FIG. 13D, a mandrel 188-1 has been inserted into the end of the tube and aligned with respect to the most proximal cut in the tube sides, after which the tube has been deformed plastically around the mandrel by applying pressure from the outside. The inwardly-displaced tube wall forms a proximal stop for a ball that is shown in FIG. 13E.

In FIG. 13F, the wall has been plastically deformed at the second most proximal cut to secure the ball in place using the ball itself as a mandrel. In alternative embodiments, this might also be achieved using glue, welding, or the like.)

In FIG. 13G, a mandrel 188-2 has been inserted into the tube and aligned with respect to the most proximal of the group of distal cuts in the sides of the tube. The tube has been plastically deformed around the mandrel to produce an inward protrusion of the wall that will serve as a barb to secure tissue.

In FIG. 13H, two more such barbs have been produced using the same or different mandrels (the final mandrel 188-4 is shown).

Finally in FIG. 13I, the tube has been cut to form slot 192 (e.g., using wire EDM) to form the two jaws of the device. Squeezing the device in the area of the aperture after tissue has been captured will then spread the jaws to allow removal of the tissue. The device as described here has no retainer to prevent premature separation of the jaws, but means of retaining the jaws can be provided if required. Since the outside of the tube is no longer smooth as a result of the deformations, and tissue may undesirably catch in external recesses, the tube may be placed coaxially within another tube, or the recesses filled in with another material, etc.

In an alternative embodiment, the first and second most proximal cuts may be oriented perpendicular to the other slots. In some embodiments, the distal end of aperture 190 may function as the most proximal slot and may be bent inward to set proximal positioning of the ball.

Numerous other alternative to this first embodiments, are possible and will be apparent to those of skill in the art.

Second Preferred Embodiment

FIGS. 14 and 15 provide perspective view from distal and proximal ends, respectively, of a device according a second embodiment of the invention. The device 202 of the second embodiment comprises a body 212 with a roughly-cylindrical proximal ‘tube interface’ section 214 intended to be inserted into the distal end of a tube as a means of handling the device; the interface section 214 of the device can be affixed to the tube by adhesive, welding, brazing, etc., or by mechanical means (e.g., catches on the device which engage features in the tube). The width, W, of the device may be on the order of 1 mm while the height, H, may be similar or considerably smaller. In other embodiments the width and height maybe smaller or larger. At the distal end of the device 202 are two hinged, spring-loaded jaws 214a and 214b with internal barbs 242a and 242b and distal cutting edges 224a and 224b; these are shown in their most spread or separated positions in the figure. Extending through the body and exiting from the proximal end is a shaft 254 which is used to control the position of the jaws. At the proximal end of the shaft is a wire coupler 234 which provides a sleeve into which a wire can be inserted and affixed (e.g., by adhesive or laser welding). The body is provided with a ceiling 240a having a tapered end 240a′ (and floor) at its distal end, which provides a closed compartment for the specimen when the jaws are retracted into the body 212. The tapered ceiling 240a′ are provided with distal cutting edges 224c and may also be provided with barbs on their inner surfaces. The floor 240b (see FIG. 18), preferably, is also provide with a distal cutting edge 224d (see, e.g., FIG. 21). Release holes 232 are provided for release of sacrificial material that may be used during an electrochemical fabrication of the device. Cutting edges 224a-224d define closed sample inlet 218 (see FIG. 21).

FIG. 16 provides a close up perspective view of the jaws of the device of FIGS. 14 and 15. The jaws 214a and 214b are provided with jaw extensions 210a and 210b and with seats 220a and 220b.

FIGS. 17 and 18 provide, respectively, perspective and plan cross-sectional views of the device of FIGS. 14-16 showing the internal springs and other details. FIG. 19 provides a cross-sectional perspective view of the distal end of the device of FIGS. 14-18. The shaft 254 is guided in its movement along the longitudinal axis of the device by proximal and distal shaft guides 260 and 262 respectively. The shaft 254 terminates at its distal end with a head 264 having holes through which pivot pins 266a and 266b attached to the jaws 214a and 214b are located. Further from the centerline of the device, the jaws are each provided with an additional pivot pin 268a and 268b which articulates with a distal end of a spring extension 270a and 270b. The proximal ends of each of the two spring extensions 270a and 270b become continuous with tension springs 272a and 272b (i.e., springs intended to be tensioned), here shown as planar meandering structures. The proximal ends of the springs are anchored to spring supports 274a and 274b which extend from the sides of the shaft 254. In this way, the tension on the springs 272a and 272b is not a function of shaft position. In other embodiments, it is, however, possible to anchor the spring to the body 212 of the device 202. As shown in the figures, ledges 276a and 276b are provided on either side of the shaft, and the jaws each have an extension that is continuous with the seat (the proximal surface) of the jaws. The device is preferably fabricated in the configuration shown in FIG. 14, such that the springs are relaxed and such that a spacing equal to or greater than a minimum feature size exists during formation of the device.

When the shaft 254 is pulled proximally, the jaws and springs slide proximally along with it, with the jaws entering the space between the floor 240b and ceiling 240a. Near the edge of the stroke the jaw seats 220a and 220b make contact with the ledges 276a and 276b respectively. Further pulling then forces the jaws 214a and 214b to rotate inwards about the jaw pivot pins 266a and 266b. This rotation stretches the springs 272a and 272b which are interfaced to the jaws via the spring extension pivot pins 268a and 268b. At the end of the stroke, the jaws have closed completely as shown in FIGS. 21 and 22 against the force of the springs 272a and 272b, such that their inner surfaces (ignoring the barbs) are substantially parallel and their outer surfaces approximating the taper of the floor and ceiling. In this configuration, the jaw seats lie against the ledges. In other embodiments, by way of tailoring the interface between the ledges and the jaw seats other jaw angles upon closure may be obtained.

FIG. 20 provides another cross-sectional perspective view of the device of FIGS. 14-19 but with the cross-section cut taken higher up in the device such that the release holes closest the ceiling are cut through as well as the upper portions of the jaws that hold the spring extensions and head onto the spring and jaw pivot pins.

FIGS. 21 and 22 provide perspective views of the distal end of the device of FIGS. 14-20 taken from different perspectives.

There are at least two modes of operation for using the device of FIGS. 14-22. In one mode, tension is applied to the shaft so that the device is initially in the configuration shown in FIGS. 21-22; the device is then pushed into the tissue to be sampled, at which time tissue enters the inlet chamber formed by the two jaws, floor, and ceiling. The device may then be withdrawn, with a specimen retained within the inlet by the barbs and torn loose from the surrounding tissue. To remove the tissue specimen from the device, the shaft is pushed distally, which both opens the jaws and pushes them to a position where the floor and ceiling are no longer blocking access to the specimen from above or below (FIG. 14).

In another operational mode, the jaws are initially spread as in FIG. 14 and the device is placed against the tissue to be sampled. By pushing the device forward while pulling on the shaft to close the jaws, a specimen is grabbed by the jaws (now acting somewhat like forceps jaws), compressed, and retained within the inlet chamber. The device is then withdrawn and the specimen removed as already described above.

Various alternatives to this embodiment are possible. Some such alternatives may involve variations similar to those discussed above in relation to the first embodiment, other alternatives may include variations discussed in association with other embodiments. In some alternative embodiments, for example, the device may take on a rectangular configuration or stepped circular approximation as opposed to the circular configuration shown.

### Third Preferred Embodiment

FIGS. 23A-23F provide a schematic representation of various stages of operation of a third embodiment of the invention. In the device of this embodiment, the tissue specimen is substantially cut free from surrounding tissue and then entrapped within a chamber in the form of a barrel by the rotation of the barrel. In FIG. 23A, the distal end 304 of the device 302 is shown with the barrel 306 in the open position where cuttings edges 312a and 314a are aligned and cutting edges 312b and 314b are aligned with the opening 316 in barrel 306 aligned with the opening 318 between cutting elements 312a and 312b. In FIG. 23B, the distal end of the device has been pushed into the tissue 310 of interest such that the cutting edges 312a, 312b, 314a and 314b penetrate into the tissue, forcing tissue to enter the interior 320 of the barrel 306. In FIG. 23C, the barrel has been rotated clockwise (as seen from the top) such that the barrel's cutting edge has severed a specimen 322 from tissue 310. In FIG. 23D, the distal end of the device has been withdrawn from the tissue 310, leaving the specimen 322 trapped within the barrel 306. In FIG. 23E, the barrel 306 has been rotated back to its open position (either by counterclockwise or clockwise rotation). Finally in FIG. 23F a push wire 324 has been passed through a hole in the proximal surface of the barrel 306 to discharge the specimen from the device. The device has some similarities with a macroscale revolving door.

In FIG. 24 provides a perspective view of an example device of that is capable of being operated according to the method of use set forth in FIGS. 23A-23F. At the distal end is an inlet port 318 with four sharpened cutting edges 314a-314d. The distal end of the device is tapered to allow for easy insertion in the target tissue while providing enough room inside the device for the barrel. In some alternative embodiments the distal end need not be tapered while in other embodiments only two of the opposing walls (i.e. floor and ceiling or left wall and right wall) may be tapered. In some embodiments, the barrel is located at the distal tip of the device, such that the tissue does not have to travel as far into the device, as is shown in this example, to enter the barrel. The device comprises a body 332 with a roughly-cylindrical proximal ‘tube interface’ section 334 intended to be inserted into a tube (not shown) as a means of handling the device. In some alternatives embodiments, the device may take on a rectangular configuration. To facilitate use at the end of a flexible endoscope or catheter, the device is designed so that barrel rotation in both directions can be achieved by applying pure tension to two shafts 342a (clockwise rotation) and 342b (counterclockwise rotation). The mechanism for producing clockwise rotation is on the top of the body of the device, while that for producing counterclockwise rotation is on the bottom of the body. The barrel is rotated clockwise by pulling on the clockwise shaft 342a. This articulates through a pin 344a, having a cap 352a that passes through a slot 354a in the top of the body 332 with a curved arm 356a. A similar but oppositely oriented cap 352b, pin 344b, slot 354b, and arm 356b exists on the bottom of the body for implementing counterclockwise movement. The arm 356a articulates at its distal end with an eccentric pin 350a attached to the top of the barrel and covered by a cap 362a. Pulling proximally on pin 350a by arm 356a causes the barrel 306 to rotate in a clockwise direction. In a similar fashion, pulling on the counterclockwise shaft causes the barrel to rotate counterclockwise by means of a similar mechanism on the bottom of the device. The design is such that the barrel need rotate much less than 180° to make the device function; thus the eccentric pins always remain on the same side of the axis of rotation of the barrel, regardless of barrel rotational position. The device may be provided with release holes such as holes 368.

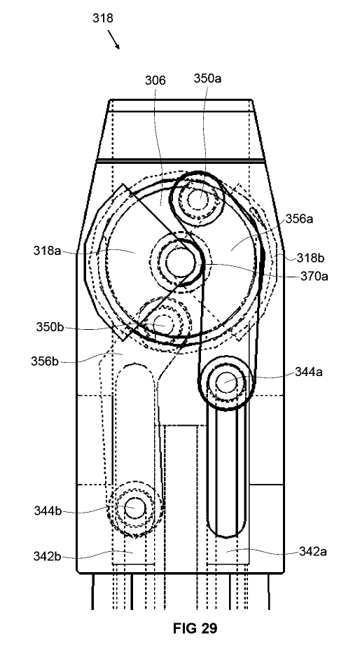

The device is preferably fabricated in the configuration shown in FIG. 24, such that the chamber is open, promoting release of sacrificial material. Release holes may be provided as necessary in any event. The barrel is articulated on central pivots (pivot 370a and 370b are visible in FIGS. 29-33), which are supported by a top plate 364a and a bottom plate, connected to the body 332. The mechanisms on top and bottom of the device may be protected from interference and/or damage due to tissue by deflection elements 366a and 366b at the top and bottom of the device which deflect the tissue over the mechanisms while the device is being inserted into tissue. The deflection elements or shields can be made to extend over the mechanisms entirely to protect them further if desired; in such a case the shields may be perforated with release holes.

FIGS. 25-30 show additional views of the device of FIG. 24 some of which are plan views, some of which are perspective views and some of which show hidden lines keeping elements that cannot be directly seen.

FIGS. 31 and 32 provide cross-sectional views through the device of FIGS. 24-30 along a plane that is perpendicular to a preferred build axis while FIG. 33 provides a cross-sectional view through the device of FIGS. 24-32 along a plane that includes the build axis and the longitudinal axis of the device. In alternative embodiments, different build axes may be chosen.

In FIG. 31, the proximal surface of the barrel is provided with an ejection hole 374 through which a push wire 324 may be passed when the opening of the barrel is centered with the inlet 318 of the body. Top and bottom guides 376a and 376b are provided for the push wire. The shafts 342a and 342b are designed to straddle the push wire without interfering with it; the push wire 324 may be inserted partway into the device while the sample is obtained, or inserted only after the device is withdrawn from the tissue, as desired. In FIG. 32, the leading edge of the barrel wall as it is advanced clockwise through the tissue is in the form of a sharp, moving blade 314b. Also visible in this figure is an optional fixed blade 314b′ on the opposite side of the device. When the barrel has sufficiently rotated, the moving and fixed blades 314b and 314b′ come together to produce a scissors-like shearing action that improves separation of the specimen from the surrounding tissue. Both blades may be made considerably sharper than is suggested by the figures.

Since, in this example, the clockwise motion involves cutting tissue and the counterclockwise motion involves merely opening the device to allow release of the specimen, the symmetric design shown in the figures may not be necessary. In other embodiments, a counterclockwise and clockwise back and forth motion may be used to cut away the specimen while minimizing the overall force exerted on the device. In some alternative embodiments the shaft and entire mechanism associated with clockwise (i.e., cutting) rotation may be made of thicker and stronger members than illustrated. In some embodiments, the mechanism for counterclockwise rotation may be omitted and the bottom mechanism designed to assist the top mechanism in producing clockwise rotation of the barrel only; in this case some other method (e.g., a separate tool) could be used to rotate the barrel counterclockwise. In some embodiments, where counterclockwise motion isn't used for cutting, a spring may be used to pull the barrel back to an aligned position with respect to the body of the device.

FIG. 34 shows a cross-sectional view of the distal end of the device as cut through the barrel pivots 370a and 370b. The barrel pivots 370a and 370b pass through holes in the top and bottom surfaces of the barrel. To reduce the clearance between the pivots and the holes such that a clearance can be reduced below a minimum feature size that may be associated with a given fabrication technology, the pivots and holes may be provided with projections 380a and 380b which are offset from one another. In addition, the outer diameter of the barrel is provided with a shoulder 378b, as is the pocket in the body of the device within which the barrel rotates 378a. The projections and shoulders are designed to allow for axial motion of the barrel on the pivots (since there is a gap between the top and bottom of the barrel and the body of the device) while still allowing the reduced clearance to be maintained. Looking at the top of the barrel, it will be noted that if the barrel rises with respect to the body, the shoulders will maintain the desired clearance, whereas if the barrel falls, the projections will maintain the clearance. Now looking at the bottom of the barrel, it can be seen this is reversed: if the barrel rises, the projections maintain the clearance, and if it falls, the shoulders maintain it. Thus the barrel is held to its axis of rotation with a reduced clearance at both its top and bottom, regardless of whether it rises or falls. Alternative configurations are possible which could also result in an effective clearance that is less than the minimum feature size.

Various alternatives to this embodiment are possible. Some such alternatives may involve variations similar to those discussed above in relation to the first and second embodiments or to be discussed hereafter in association with other embodiments. In some alternative embodiments, for example, if the compression strength of the shafts and the interface tube are insufficient to hold the device in place while the barrel is rotated, an additional, more compressionally rigid shaft may be attached to the proximal end of the interface tube.

### Fourth Preferred Embodiment

FIG. 35 shows a partial view of a distal end of a device according to a fourth embodiment of the invention. This device 402 is similar in some ways to that of the device 302 of the third embodiment, in that the tissue specimen is substantially cut free from surrounding tissue and then entrapped within a chamber after passing through inlet 418 and the door closing. Here, however, in lieu of a rotating barrel there is a door 404 divided into articulated strips 406a-406q, the leading edge 408 of strip 406 of which forms a moving blade 408 that can cut a specimen loose that is located within the space behind the door as the door closes; the specimen then becomes trapped behind the closed door until it is later opened. The door resembles a ‘roll-up’ door of the kind used in industrial buildings, but on a much smaller scale. Each of the strips is able to flex relative to its neighbors by means of compliant or pivoting joints between them (not shown), thus allowing the door to bend around corners (i.e. change direction). Attached to the leading edge of the door is at least one chain 412a; pulling on this chain closes the door, whereas pulling on the opposite end 414 of the door opens it. In some embodiments additional chains, e.g. 412b, can be added. The door 404 and chain 412a can be attached to wires through suitable couplers (as in the device of the second embodiment) allowing the device to be actuated remotely (e.g., if delivered through an endoscope or at the end of a catheter). Pulleys 414 (or sprockets, if the door and chain have recesses to accommodate the teeth) are provided to guide the door and chains around the corners. A fixed blade (not shown) can also be provided to produce a scissors-like cutting action at the far edge of the door jam (not shown) when the door is closed.

Various alternatives to this embodiment are possible. Some such alternatives may involve variations similar to those discussed above in relation to the first and second embodiments or to be discussed hereafter in association with other embodiments.

### Additional Alternatives and Enhancement for the Previously Discussed Embodiments

In variations of the devices of the first four embodiments, stops or flanges on the outside surface of the devices may be included to prevent the devices from being inserted too far into the tissue. These stops may be adjustable in position. The devices may be interfaced to catheters or handles, passed through endoscope working channels, and the like. Alternatively, the devices may be directly pushed into the tissue to be sampled. Alternatively, in some embodiments the devices may be attached to moving shuttles which are launched into the target tissue from some initial standoff distance. A spring mechanism, pneumatics, and other means may be used to launch the shuttle toward the tissue with enough momentum to achieve penetration of the tissue to the required depth. Potential advantages of obtaining biopsy specimens using launching in lieu of pushing include the ability to deliver the device to the tissue using a more compliant device (such as a flexible endoscope) than would otherwise be practical, and reduction in pain associated with biopsy (penetration of the device into the tissue can be much faster). In some embodiments, a chain, wire, or similar structure can be attached to the shuttle to facilitate retrieval of the device once it has penetrated. In the case of the devices of the third and fourth embodiments, rotation of the barrel or closing of the door can be achieved by pulling on a chain or wire to actuate the barrel or door before pulling the device out of the tissue.

Some Advantages of the Methods and Devices of the First Four Embodiments Over Traditional Techniques for Extracting Specimens:

The devices of the first four embodiments may be used in lieu of commonly-used needles for fine needle aspiration (FNA), and existing devices for core biopsy. The former comprise hollow needles to which is attached a vacuum source; when the needle is inserted into the tissue to be biopsied, vacuum is applied and tissue and fluid are withdrawn. The latter devices typically are needles having a side port through which tissue can enter, either by itself or with the assistance of vacuum applied through the device. The tissue which has entered the device is then excised and retained in the device which is then withdrawn; typically a sliding cutting blade within the device is used for excision. The devices of the first four embodiments have these advantages over FNA needles:
Intact tissue specimens suitable for histological—rather than merely cytological analysis can be obtained with a small diameter device. Histological examination typically allows a more reliable diagnosis since more structure is preserved.
Specimens can be obtained more reliably; the barbs grab tissue securely, holding onto it well enough that withdrawal of the device tears the tissue loose for examination. This is to be contrasted with FNA needles which rely on limited suction force and with which it is not uncommon to need multiple needle penetrations or manipulations in order to obtain a good specimen.

The devices of the first four embodiments have these advantages over core biopsy devices:
Smaller diameter reduces tissue trauma, pain, and recovery time. If the smaller volume of tissue obtained with a smaller diameter device is inadequate, multiple insertions/withdrawals (full or partial) may be used.
More accurate targeting of the desired specimen. Since the inlet to the device is at the distal end, rather than on the side, it is easier to aim the device, and there is no ambiguity as to which side of the device the port is located.

### Fifth Preferred Embodiment

FIGS. 36-37 show a partial view of a fifth embodiment of the invention. In this embodiments two opposing cups are closed about a sample to trap and contain a specimen that is to be extracted from the body. In the views provided half the housing, as well as one of two tissue cups have been removed to better illustrate the operational mechanism. In addition, most of the teeth on the gears, pinions, and racks are not shown. Inside the housing is a shaft which may be attached to a wire or cable. Pulling the shaft brings the two tissue cups toward one another by means of a gear train.

The device is intended to overcome a significant problem with conventional cup biopsy forceps used in endoscopic procedures. Such forceps typically have tissue cups which are fastened to each other through a pivot near their proximal ends. The cups have long proximal extensions which extend proximally to the pivot, to the ends of which are attached articulating linkages through pins. The linkages are in turn attached to a central cable or wire. When the wire is pushed distally, the cups separate, and when it is pulled, the cups come together. The problem is that when the cups are separated, the extensions of the cups and the linkages increase the cross-sectional area of the device dramatically, and often block the view of the tissue to be sampled (as viewed through the endoscope). The present device is designed to maintain a small cross-section (other than the cups themselves), affording an improved view of the target tissue. It is not sufficient, however, to achieve this goal by merely shortening the length of the cup extensions and the linkages, since one would then lose significant leverage on the cups, and thus not be able to exert sufficient force to cut loose the tissue when the cups are brought together. The use of a gear train to transform a long axial movement of the shaft applied with a fairly low tension, into a short, high-torque rotation of the cups, is a key aspect of the device. In another embodiment, a set of hinged linkages are used to achieve this same force amplification.

In the figures, the tissue cups 512 are at the distal end of a housing 504 of the device 502. The cups 512 are pivoted near their proximal ends and the pivots incorporate pinions 514 (with teeth (not shown) that do not necessarily cover the full 360° circumference of the pinions) which are made to rotate by means of a toothed rack 516 (teeth not shown) passing between the pinions. Pulling on the rack (toward the proximal end of the device) causes the cups to rotate inwards, to cut and retain a biopsy specimen. The rack 516 is attached to a block 518 which is provided with guides (not shown) to guide its distal/proximal motion. At the proximal end of the block is a second rack 522 which engages a pinion 524. Rotation of this pinion 524 causes the rack 522, block 524, and the cups 512 to move. Pinion 524 is actually the final gear of a gear train that has been designed to provide the required force amplification. The most proximal gear 532 of the gear train is also a pinion which engages a third rack 534, which is attached to a shaft 542 which is used by a surgeon or other operator to actuate the cups. The rack 534 is relatively long (since a long stroke of the shaft must be accommodated).

### Sixth Preferred Embodiment

FIG. 38 shows a device of a sixth embodiment of the invention. The device of this embodiment is similar in some ways to a macroscale ice-cream scoop, but on a much-reduced scale. The device shown in the figure is not to scale, and may incorporate other elements (e.g., a fixed or retractable housing that surrounds the scoop). The device 602 is inserted into the tissue to be sampled and the shaft 618 is rotated in the direction 614 shown (e.g. approximately 360°) such that the cutting edge 616 of the scoop 620 cuts out a portion of the tissue, which then fills the scoop. If desired, the vane 624 may then be rotated approximately 90° in direction 626 relative to the body of the scoop in order to help retain the specimen within the scoop. The device is then withdrawn and the vane is rotated in the direction opposite to 626 via control rod 628 to release the specimen; this may involve rotating it so that it passes between the specimen and the inner surface of the scoop, much like the vane of an ice cream scoop is used to release ice cream from the scoop.

In some embodiments, the vane may be made in a different shape: that of a smaller diameter, somewhat hemispherical inner scoop fitting within the outer scoop shown in the figure, and equipped with a sharpened leading edge. In this case, the inner scoop is rotated about 180 relative to the outer scoop which may complete the cut. Alternatively different relative movements of the inner and out cups may be used to complete the cutting and separation of the sample from the remaining tissue. When cutting is completed, the two scoops together substantially surround the specimen, capturing it. At this time, the device is withdrawn and the inner scoop is rotated back relative to the outer scoop to release the specimen.

### Seventh Preferred Embodiment

The device of this embodiment is similar in some ways to a macroscale cheese grater, but on a much smaller scale. It may be suitable for shave-type biopsies of surfaces such as the skin, or for thinning tissue (e.g., excess tissue causing thickening of the aortic valve of the heart). Sharpened projections 712 on device 702 cut tissue when the device is slid along the surface of the tissue, and the tissue that is cut enters the holes 714 in the surface of the device. The device may be provided with vacuum and/or irrigation fluid so as to extract specimens. In some embodiments, no holes are provided, and the tissue remains on the same side of the device as the projections, until removed.

### Additional Alternatives and Conclusions

In some embodiments the formation of the devices described herein via multilayer electrochemical fabrication processes may be include various additional processing steps to improve the reliability or functionality of the devices. For example, some embodiments may employ diffusion bonding or the like to enhance adhesion between successive layers of material. Various teachings concerning the use of diffusion bonding in electrochemical fabrication process are set forth in U.S. Patent Application No. 60/534,204 which was filed Dec. 31, 2003 by Cohen et al. which is entitled “Method for Fabricating Three-Dimensional Structures Including Surface Treatment of a First Material in Preparation for Deposition of a Second Material”; U.S. patent application Ser. No. 10/841,382, filed May 7, 2004 by Zhang, et al., and which is entitled “Method of Electrochemically Fabricating Multilayer Structures Having Improved Interlayer Adhesion”; U.S. patent application Ser. No. 10/841,384, filed May 7, 2004 by Zhang, et al., and which is entitled “Method of Electrochemically Fabricating Multilayer Structures Having Improved Interlayer Adhesion”. Each of these applications is incorporated herein by reference as if set forth in full.

The formation of the alternative devices may involve use of structural or sacrificial dielectric materials. Additional teachings concerning the formation of structures on dielectric substrates and/or the formation of structures that incorporate dielectric materials into the formation process and possibility into the final structures as formed are set forth in a number of patent applications filed Dec. 31, 2003. The first of these filings is U.S. Patent Application No. 60/534,184 which is entitled “Electrochemical Fabrication Methods Incorporating Dielectric Materials and/or Using Dielectric Substrates”. The second of these filings is U.S. Patent Application No. 60/533,932, which is entitled “Electrochemical Fabrication Methods Using Dielectric Substrates”. The third of these filings is U.S. Patent Application No. 60/534,157, which is entitled “Electrochemical Fabrication Methods Incorporating Dielectric Materials”. The fourth of these filings is U.S. Patent Application No. 60/533,891, which is entitled “Methods for Electrochemically Fabricating Structures Incorporating Dielectric Sheets and/or Seed layers That Are Partially Removed Via Planarization”. A fifth such filing is U.S. Patent Application No. 60/533,895, which is entitled “Electrochemical Fabrication Method for Producing Multi-layer Three-Dimensional Structures on a Porous Dielectric”. Additional patent filings that provide teachings concerning incorporation of dielectrics into the EFAB process include U.S. patent application Ser. No. 11/139,262, filed May 26, 2005 by Lockard, et al., and which is entitled “Methods for Electrochemically Fabricating Structures Using Adhered Masks, Incorporating Dielectric Sheets, and/or Seed Layers that are Partially Removed Via Planarization”; and U.S. patent application Ser. No. 11/029,216, filed Jan. 3, 2005 by Cohen, et al., and which is entitled “Electrochemical Fabrication Methods Incorporating Dielectric Materials and/or Using Dielectric Substrates”. These patent filings are each hereby incorporated herein by reference as if set forth in full herein.

Further teachings about planarizing layers and setting layers thicknesses and the like are set forth in the following U.S. Patent Applications which were filed Dec. 31, 2003: (1) U.S. Patent Application No. 60/534,159 by Cohen et al. and which is entitled “Electrochemical Fabrication Methods for Producing Multilayer Structures Including the use of Diamond Machining in the Planarization of Deposits of Material” and (2) U.S. Patent Application No. 60/534,183 by Cohen et al. and which is entitled “Method and Apparatus for Maintaining Parallelism of Layers and/or Achieving Desired Thicknesses of Layers During the Electrochemical Fabrication of Structures”. An additional filings providing teachings related to planarization are found in U.S. patent application Ser. No. 11/029,220, filed Jan. 3, 2005 by Frodis, et al., and which is entitled “Method and Apparatus for Maintaining Parallelism of Layers and/or Achieving Desired Thicknesses of Layers During the Electrochemical Fabrication of Structures”. These patent filings are each hereby incorporated herein by reference as if set forth in full herein.

Many other alternative embodiments will be apparent to those of skill in the art upon review or the teachings herein. Further embodiments may be formed from a combination of the various teachings explicitly set forth in the body of this application. Even further embodiments may be formed by combining the teachings set forth explicitly herein with teachings set forth in the various applications and patents referenced herein, each of which is incorporated herein by reference. In view of the teachings herein, many further embodiments, alternatives in design and uses of the instant invention will be apparent to those of skill in the art. As such, it is not intended that the invention be limited to the particular illustrative embodiments, alternatives, and uses described above but instead that it be solely limited by the claims presented hereafter.

I claim:

1. A medical device for extracting a specimen of tissue from the body of a patient, comprising:
(a) means for allowing a specimen to access an opening within the device;
(b) means for holding the specimen within the opening;
(c) means for separating the specimen from other tissue; and
(d) means for releasing the specimen from the opening.

2. The instrument of claim 1, wherein the instrument includes a plurality of layers of deposited material which were substantially patterned and adhered to previously formed layers at the time of deposition.

3. The instrument of claim 1 wherein the means for allowing a specimen to access an opening within the device is an inlet having a wall with at least one edge of the wall sharp to cut tissue as the instrument is inserted into a tissue region from which the specimen is to be extracted.

4. The instrument of claim 1 wherein the means for holding the specimen within the opening comprising a plurality of barbs.

5. The instrument of claim 4 wherein the means for holding the specimen within the opening comprises jaws which can clamp down on the specimen.

6. The instrument of claim 1 wherein the means for holding the specimen within the opening comprises Barrel which can be rotated to a position to substantially trap the sample within the opening.

7. The instrument of claim 1 wherein the means for holding the specimen comprises a ceiling and floor which cap the top and bottom of at least a left jaw and a right jaw.

8. The instrument of claim 1 wherein the means for separating comprising a means for cutting

9. The instrument of claim 9 wherein the means for cutting comprises a scissor-like motion between blades that move past one another.

10. The instrument of claim 1 wherein the means for separating comprising the means for holding and a means for moving the device away from other tissue to which the specimen is attached so as to separate the specimen from the other tissue.

11. The instrument of claim 1 wherein the means for holding comprises a pair of jaws and the means for releasing comprises means for opening the jaws.

12. A method for extracting a tissue specimen from a body of a patient, comprising:
(a) locating an extraction device in proximity to the tissue to be sampled;
(b) causing a specimen of tissue to enter an opening within the extraction device;
(c) causing the device to hold the specimen within the opening;
(c) causing the specimen to separate from other tissue; and
(d) releasing the specimen from the opening.
